(19) United States
(12) Patent Application Publication
(10) Pub. No.: US 2024/0165301 A1
(43) Pub. Date: May 23, 2024

(54) CELL AND COLLAGEN COMPOSITIONS FOR ENGINEERED CARDIAC TISSUE

(71) Applicant: BROWN UNIVERSITY, Providence, RI (US)

(72) Inventors: Kareen Coulombe, Providence, RI (US); Kiera Dwyer, Providence, RI (US); Rajeev Kant, Providence, RI (US)

(21) Appl. No.: 18/513,051

(22) Filed: Nov. 17, 2023

Related U.S. Application Data
(60) Provisional application No. 63/426,287, filed on Nov. 17, 2022.

(51) Int. Cl. A61L 27/38; A61L 27/24; A61L 27/52; C12N 5/077

(52) U.S. Cl. CPC A61L 27/3873; A61L 27/24; A61L 27/3826; A61L 27/52; C12N 5/0656; C12N 5/0657; A61L 2430/20; C12N 2506/45; C12N 2533/54

(57) ABSTRACT

Compositions for generating cardiac tissue are provided. The compositions may improve cardiac tissue function.

### CROSS REFERENCE TO RELATED APPLICATIONS

This application claims the benefit of U.S. Provisional patent application 63/426,287 filed Nov. 17, 2022, the entire contents of which are incorporated by reference herein.

### STATEMENT REGARDING FEDERALLY SPONSORED RESEARCH OR DEVELOPMENT

This invention was made with government support under Grant Nos. HL135091, F32HL160063, T32 GM065085, NIH RO1HL128831, and RO1HL46716 awarded by the National Institute of Health and Grant No. P20GM103652 awarded by National Institute of Health Cardiovascular Pulmonary Vascular Biology. The government has certain rights in the invention.

### FIELD

The present disclosure relates to compositions for generating or aiding in the generation of cardiac tissue.

### BACKGROUND

Cardiovascular disease (CVD) is the leading cause of death worldwide. CVD can lead to myocardial infarction (MI), also known as a “heart attack,” which results in restricted blood flow and extensive cell death within the infarct zone. Due to the limited regenerative capacity of the human heart, infarcted myocardium is replaced by fibrotic scar tissue with inferior contractile performance. Over time, pathological remodeling leads to ventricular wall thinning, which can progress to heart failure. There is currently no treatment available that can restore lost cardiomyocytes after MI, and conventional therapies typically only manage the symptoms.

It is estimated that one billion cardiomyocytes are killed during a myocardial infarction (MI). Ongoing work in the field of cardiac regenerative engineering is aimed at developing strategies to restore both cellular and functional loss from MI. Delivery of cardiomyocytes (CMs) derived from human embryonic or human induced pluripotent stem cells (hiPSC-CMs) through engineered tissue or intramuscular injection after such injury has emerged as a promising means to stabilize cardiac function. Delivery of CMs through epicardially-implanted engineered cardiac tissues (ECTs) is especially advantageous as increased CM maturity, improved engraftment and increased mechanical support of the cardiac wall have been reported as compared to intramuscular CM injection, and new data suggests lower incidence of arrhythmia. To date, however, a lack of methods for robust manufacturing of ECTs poses an immense hurdle to their use in the clinic.

There is a need for protocols that allow for scaling up ECTs in size, CM number or density, and for controlling the dose of CMs. There is a need for approaches that can restore the function of a patient's heart and replace or regenerate infarcted myocardium.

### SUMMARY

Provided is an engineered cardiac tissue (ECT) construct comprising cardiomyocytes (CMs) and having a CM density of about 5 million CMs/mL to about 75 million CMs/mL. The ECT comprises up to 1 billion cells. The present disclosure details the differentiation and proliferative expansion of hiPSC-CMs to achieve the quantity and quality of hiPSC-CMs required for clinically scaled ECTs. ECTs were fabricated by mixing hiPSC-CMs with 5% human cardiac fibroblasts in a collagen-1 hydrogel with electromechanical function assessed through optical mapping of action potentials and tensile mechanical testing. Because such tissues must deliver enough CMs to have a therapeutic impact, the present disclosure explores increasing cell density in ECTs, reporting decreased compaction during formation (6-fold increase, assessed by cross-sectional area), elastic modulus (20-fold decrease) and active stress generation (6.5-fold decrease) with increasing cell density from 5 M/mL to 50 M/mL. By understanding the design space and functional changes, the present disclosure supports the clinical feasibility of using hiPSC-CMs within ECTs as a regenerative treatment to remuscularize the failing heart.

### BRIEF DESCRIPTION OF THE DRAWINGS

FIG. 1 shows ECT scaleup. Meso-, macro-, and mega-ECTs with densities and cell quantities. K refers to thousand; M refers to million; and B refers to billion.

FIGS. 2A-2B show preclinical swine model of chronic myocardial ischemia. FIG. 2A shows the timeline of in vivo swine study. FIG. 2B shows a schematic of ameroid constrictor placement on the proximal left circumflex (LCX) coronary artery to induce chronic myocardial ischemia in swine model.

FIGS. 3A-3D show increasing hiPSC-CM dose within meso-tissues decreases mechanical integrity and structural survival during culture. FIG. 3A shows Brightfield images illustrating tissue compaction from initial casting on day 0 (D0) to day 7 (D7) of in vitro culture (mold size, 3×9 mm); FIG. 3B shows survival curve showing percentage of intact meso-ECTs to assess structural survival; FIG. 3C shows quantification of tissue compaction over 7-day culture; FIG. 3D shows elastic modulus of tissues to assess passive mechanical properties with each point representing a single meso-ECT. 1 mg/mL collagen concentration utilized; M indicates million; n=15-28 samples per group with significance defined as *p<0.5; ***p<0.001, ****p<0.0001 with statistically significant comparison present between 5 M/mL and all other groups and 15 M/mL with all other groups; # indicates all conditions except the comparison between 5 M/mL and 15 M/mL are significant with a minimum p<0.05; $ indicates all conditions are significant to each other at a minimum p<0.05.

FIGS. 4A-4G show structural integrity observed in different meso-ECT dose conditions is correlated with developed prestrain at the tissue level as well as extent and organization of collagen remodeling during tissue formation at the cellular level. FIG. 4A shows Brightfield images of ECTs showing curling in stress-free environment (removed from posts) after 7 days of culture; FIG. 4B shows quantification of ECT curling angle (degrees) and FIG. 4C shows developed prestrain (%); FIG. 4D shows representative histological staining of PRFG for each density condition; FIG. 4E shows quantification of the picrosirius red (collagen content) and fast green (basic proteins, cytoplasm content) per condition measured by % of area analyzed; FIG. 4F shows representative images of second harmonic generation (SHG) imaging showing organized collagen fibrils; FIG. 4G shows quantification of collagen content imaged through SHG for each condition, measured by % area analyzed. 1 mg/mL collagen concentration utilized; M indicates million; n=7-10 analyzed tissues per group for Picrosirius Red, Fast Green (PRFG) staining; n=12-23 tissues analyzed for curling angle and prestrain quantification per condition; n=4-7 for SHG with multiple areas averaged to analyze per tissue; the individual points represent single meso-ECTs analyzed; *p<0.5; **p<0.01; ***p<0.001, ****p<0.0001.

FIGS. 5A-5E show increasing hiPSC-CM density within meso-ECTs alters normalized force generation, with histomorphological analysis confirming reduced sarcomere organization. FIG. 5A shows active stress generation of ECTs from 0-30% stretch, measured at increments of 5%; FIG. 5B shows active stress, FIG. 5C shows force, and FIG. 5D shows force normalized by the number of hiPSC-CM within the ECTs at 30% stretch; FIG. 5E shows histological staining of α-sarcomeric actinin (α-actinin), wheat germ agglutinin (WGA), and Hoechst. N=15-24 tissues analyzed mechanically per condition; 1 mg/mL collagen concentration utilized; M indicates million; n=7-13 tissues analyzed for histology with multiple regions averaged per tissue for each condition; the individual points represent single meso-ECTs analyzed; *p<0.01; ***p<0.001, ****p<0.0001.

FIGS. 6A-6G show macro-ECTs maintain dose dependence of tissue survival, collagen content, and organization but have more uniform compaction. FIG. 6A shows Brightfield images show macro-tissue compaction at day 7 (D7) of in vitro culture (mold size, 8×12 mm); FIG. 6B shows survival curve of percentage of intact tissues shows structural integrity begins to decline on day 4; FIG. 6C shows quantification of tissue compaction over 7-day culture; FIG. 6D shows representative histological staining of PRFG for each density condition; FIG. 6E shows quantification of the picrosirius red (collagen content) and fast green (basic proteins, cytoplasm content) per condition, measured by % of area analyzed; FIG. 6F shows representative images of SHG imaging showing organized collagen fibrils, measured by % area analyzed; FIG. 6G shows quantification of collagen content imaged through SHG for each condition. Collagen concentration of 3.5 mg/mL utilized; M indicates million; n=9-10 tissues per condition analyzed for formation; n=4-6 tissues analyzed histologically with multiple regions averaged per tissue for each condition; the individual points represent single macro-ECTs analyzed; *p<0.5; **p<0.01; ****p<0.0001.

FIGS. 7A-7E show increased cell density within macro-tissue format shows limited sarcomere organization but increased contractile amplitude and ability for electrical pacing with no arrhythmia generation. FIG. 7A shows histological staining of α-sarcomeric actinin (α-actinin), wheat germ agglutin (WGA), and Hoechst; FIG. 7B shows video-based analysis of ECT contractility using the software MUSCLEMOTION® to quantify contraction amplitude; FIG. 7C shows percent of tissues following 0.5 Hz point stimulation pacing during optical mapping; FIG. 7D shows heatmap of activation sequences generated from GCaMP calcium transient recordings for 15 M/mL macro-ECT (left) and 50 M/mL macro-ECT (right); FIG. 7E shows maximum capture rate (MCR) of macro-ECTs under field stimulation. Collagen concentration of 3.5 mg/mL utilized; M indicates million; n=4-6 tissues analyzed with multiple regions averaged per tissue for each condition for histological analysis; n=9-10 tissues per condition analyzed for contractility; n=5-6 tissues per condition attempted for optical mapping, with pacing achieved in n=2-5; the individual points represent single macro-ECTs analyzed; *p<0.5; **p<0.01.

FIGS. 8A-8I show scale-up to clinical-sized engineered tissue allows for the delivery of 1 billion hiPSC-CMs within a single tissue. FIG. 8A shows Brightfield images illustrating mega-tissue compaction during in vitro culture (mold size, 65×75 mm) and tissue under dynamic culture on a rocker; FIG. 8B shows video-based analysis of ECT contractility using the software MUSCLEMOTION® to quantify contraction amplitude at the external post of the mega-ECT; FIG. 8C shows quantification of tissue compaction over in vitro culture; FIG. 8D shows representative cross-sectional PRFG stain from the middle of the mega-ECT pre-implantation; FIG. 8E shows cross-section histological staining of the full transverse of the middle portion of the mega-ECT with hiPSC-CM marker, cTnT pre-implantation; FIG. 8F shows histological staining of myosin light chain 2, specifically MLC2a which represents the immature isoform and MLC2v which represents the more mature isoform pre-implantation. FIG. 8G shows Implantation of mega-ECTs on epicardial surface of swine model of chronic ischemia model; FIG. 8H shows histological staining of mega-ECT post-implantation with cTnT, human-specific nuclear marker (hu-Ku80), and Hoechst with section taken from the anterior-lateral basal region of the heart; FIG. 8I shows histological staining of mega-ECT post-implantation with MLC2a, MLC2v, and Hoechst. n=4 mega-ECTs fabricated and assessed for structural survival, compaction, and contractility with section taken from the anterior-lateral basal region of the heart. 50 M/mL hiPSC-CM density and 3.5 mg/mL collagen concentration utilized; M indicates million; n=4 mega-ECTs fabricated; n=2 implanted mega-ECTs in swine model; n=1 analyzed for histology with multiple regions averaged for all quantification.

FIG. 9 shows electrophysiology of ECTs having 5-50% CFs.

FIG. 10A shows a representation of fraction of beats in ECTs following 1 Hz stimulation for 5%, 10% and 15% CFs. FIG. 10B shows a representation of a fluorescence image of paced beats from 5% hCF ECT, stimulated from the left upper corner. FIG. 10C shows a representation of sample action potential traces from the locations marked with “A,” “B,” and “C” in FIG. 10B.

### DETAILED DESCRIPTION

Moving ECTs towards clinical implementation requires advanced biomanufacturing and tissue engineering approaches to handle the large number of PSC-CMs that would need to be delivered and/or grafted in the clinic. When ECTs are scaled up using size alone as the metric, compromised electromechanical function and structural organization of ECTs are reported. Another consideration is the cell dose that can be delivered by direct intramuscular injection or epicardially within engineered cardiac tissue (ECT), because the dose has a potential impact on cardiac function. Injected PSC-CMs can produce arrhythmias, including ventricular tachycardia. By contrast, implanted ECTs develop an electrical syncytium, are structurally whole upon implantation, have a lower arrhythmia risk, and their development is supported by some preclinical studies. It is estimated that about one billion cardiomyocytes are killed during a myocardial infarction (MI). An ongoing challenge in the field is the need for up to 1 billion cells to be incorporated in an implant volume that is small enough to be amenable to surgical implantation in living patients. The space limitations within the thoracic cavity add to the challenges of implanting ECTs. Thus a higher density of ECTs is desirable, and further the higher density of ECTs should be capable of mimicking host cardiac tissue. Further, the implanted ECTs should be capable of electromechanically integrating with host cardiac tissue and promoting functional recovery.

Recent work in the field supports the notion that PSC-CM treatment has a therapeutic window, or dose of cells, that improves cardiac function after injury. Using a cryoinjury guinea pig model of MI, ECTs were implanted with a low (4.5 M), medium (8.5 M), or high (12 M) hiPSC-CM dose in vivo, finding a dose-dependent effect on remuscularization of the scar (low: 1.4±0.4%, medium: 5.3±2.0%, high: 12.3±2.5%) as well as functional improvement assessed by left ventricular (LV) fractional area shortening (FAS), with only the highest dose having a significant impact (+8% absolute, +24% relative increase). Upscaling these results to patients, it is estimated that ˜1000×106 (1 billion) CMs are required to have a therapeutic impact on the post-MI adult human heart. Despite the importance of scaling up ECTs in terms of cell dose to maximize therapeutic impact as well as size for clinical relevance, the design parameters for manufacturing cell-dense ECTs that can accommodate the 1 billion CMs lost during MI in patients have not been adequately defined, nor are they reliably reproducible. Multiple interconnected factors such as cell density, size, extracellular matrix concentration, and time impact tissue formation, function, and potential treatment efficacy, can affect the outcome and ECT scale up is typically not amenable to linear scale up. Accordingly described herein are certain design spaces for fabricating ECTs at possibly clinically relevant cell doses and sizes and provided are some solutions for the gaps that emerge upon scaling up.

A goal of cell-based cardiac regeneration therapies is to remuscularize the heart and subsequently improve cardiac function after injury. Preclinical models have shown engraftment of pluripotent stem cell derived cardiomyocytes (PSC-CMs) in diseased hearts, including improved contractile function with engraftment. Like any treatment, however, dose is a key factor impacting therapeutic efficacy. A dependence of remuscularization on the dose of hiPSC-CMs implanted in vivo has been reported; yet, the appropriate cell dose required for maximal functional benefit after cardiac injury remains unknown. In the case of myocardial infarction (MI), it is likely this therapeutic dose is on a spectrum, dependent on factors such as disease severity (infarct size, location, etc.), progression of healing (remodeling, dilation, time between injury and intervention, etc.), as well as patient demographics (age, anatomy, response to treatment, comorbidities, co-therapies, etc.). The methods described herein may allow for tuning cell dose within ECTs to accommodate patient variability and disease state.

Typically the hydrogel matrix for ECTs has been fibrin or collagen. Fibrin can be prepared at much higher concentrations than collagen and also gels quickly by addition of thrombin. However, fibrin gels also degrade quickly due to enzymatic degradation and can cause clotting when used in ECTs. Collagen gels slowly, but is the predominant structural protein in native myocardium. Accordingly, described herein are collagen gels having a higher density of collagen per milliliter compared to previous studies with ECTs. Advantageously, a higher collagen gel density, as used herein, imparts robust structure to the macro and mega ECTs described herein while also retaining functional capability of the ECT. Further, use of casting solutions comprising ratios of CMs to collagen gel, as described herein, allows for scaling meso ECTs to mega and macro ECTs.

It has also been found that including human cardiac fibroblasts in the ECTs at concentrations described herein improves tissue formation, material properties, and contractile functions, compared to ECTs prepared without any human cardiac fibroblasts.

### Definitions

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

“CM density” means the number of cardiomyocytes per mL.

“Animal” includes human and non-human species such as rats, mice, dogs, cats, rabbits, and primates.

As used herein, the terms “individual”, “patient”, or “subject” used interchangeably, refer to any animals, including mammals, preferably mice, rats, other rodents, rabbits, dogs, cats, swine, cattle, sheep, horses, or primates, and most preferably humans.

As used herein, “the number of CFs is about X % of the number of CMs” means that CF cell count is about X % of the cell count for the CMs. By way of example only, in some embodiments, when about 1 billion CMs are in a composition, about 50 million CFs are included in the composition for a total of about 1.05 billion cells in the composition.

“Implanting (or implantation of) an ECT” means surgically introducing said ECT in an affected area (e.g., the myocardium). Implanting includes and is not limited to transplantation, intramyocardial injection, and/or attachment of epicardial patches.

In some embodiments, “substantially comprised of collagen” means at least 80% of the gelling component in the hydrogel is collagen. In some embodiments, “substantially comprised of collagen” means at least 85% of the gelling component in the hydrogel is collagen. In some embodiments, “substantially comprised of collagen” means at least 90% of the gelling component in the hydrogel is collagen. In some embodiments, “substantially comprised of collagen” means at least 95% of the gelling component in the hydrogel is collagen.

An aspect of the present disclosure is directed to a method for improving cardiac tissue survival. The method includes preparing a composition that includes cardiomyocytes at a density of about 5 to about 75 M/mL and a collagen hydrogel at a concentration of about 1 to about 3.5 mg/mL, and administering the composition to a subject in need thereof.

In some embodiments, the composition is administered epicardially to the subject.

In some embodiments, the method further includes monitoring and assessing cardiac tissue survival in the subject.

In some embodiments, the method reduces the incidence of arrhythmia.

In some embodiments, the method increases mechanical support of the cardiac wall.

In some embodiments, the collagen hydrogel is derived from rat tail collagen.

Another aspect of the present disclosure is directed to a method for fabricating engineered cardiac tissue. The method includes combining human induced pluripotent stem cells (hiPSC-CMs), primary human cardiac fibroblasts, and collagen hydrogel, and culturing the engineered cardiac tissue to achieve a desired electromechanical function.

In some embodiments, the collagen hydrogel is at a concentration of about 1 to about 3.5 mg/mL.

In some embodiments, the collagen hydrogel is derived from rat tail collagen.

Provided herein is an engineered cardiac tissue (ECT) composition comprising cardiomyocytes (CMs) and having a characteristic CM density.

In some embodiments, the engineered cardiac tissue (ECT) composition comprises up to about 1 billion cardiomyocytes (CMs).

In other embodiments, the engineered cardiac tissue (ECT) composition comprises cardiomyocytes (CMs) and has a CM density of about 5 million CMs/mL to about 75 million CMs/mL.

In still other embodiments, the engineered cardiac tissue (ECT) composition comprises up to about 1 billion cardiomyocytes (CMs) and has a CM density of about 5 million CMs/mL to about 75 million CMs/mL.

In some embodiments, the ECT composition has a CM density of about 15 million CMs/mL to about 75 million CMs/mL. In some embodiments, the ECT composition has a CM density of about 17 million CMs/mL to about 75 million CMs/mL. In some embodiments, the ECT composition has a CM density of about 20 million CMs/mL to about 75 million CMs/mL. In some embodiments, the ECT composition has a CM density of about 25 million CMs/mL to about 75 million CMs/mL. In some embodiments, the ECT composition has a CM density of about 30 million CMs/mL to about 75 million CMs/mL. In some embodiments, the ECT composition has a CM density of about 35 million CMs/mL to about 75 million CMs/mL. In some embodiments, the ECT composition has a CM density of about 40 million CMs/mL to about 75 million CMs/mL. In some embodiments, the ECT composition has a CM density of about 50 million CMs/mL to about 75 million CMs/mL. In some embodiments, the ECT composition has a CM density of about 20 million CMs/mL to about 70 million CMs/mL. In some embodiments, the ECT composition has a CM density of about 25 million CMs/mL to about 65 million CMs/mL. In some embodiments, the ECT composition has a CM density of about 25 million CMs/mL to about 60 million CMs/mL. In some embodiments, the ECT composition has a CM density of about 25 million CMs/mL to about 55 million CMs/mL. In some embodiments, the ECT composition has a CM density of about 25 million CMs/mL to about 50 million CMs/mL. In some embodiments, the ECT composition has a CM density of about 30 million CMs/mL to about 50 million CMs/mL. In some embodiments, the ECT composition has a CM density of about 40 million CMs/mL to about 50 million CMs/mL.

In some embodiments, the ECT composition has a CM density of about 50 million CMs/mL. In some embodiments, the cardiomyocytes in the ECT composition are at a density of about 1 M/mL, about 5 M/mL, about 10 M/mL, about 15 M/mL, about 20 M/mL, about 25 M/mL, about 30 M/mL, about 35 M/mL, about 40 M/mL, about 45 M/mL, about 50 M/mL, about 55 M/mL, about 60 M/mL, about 70 M/mL, about 75 M/mL, about 80 M/mL, about 85 M/mL, about 90 M/mL, or about 95 M/mL. As used herein, M/mL refers to million CMs/mL

In some embodiments, the cardiomyocytes are human induced pluripotent stem-cell derived cardiomyocytes (hiPSC-CMs) or human embryonic stem-cell derived cardiomyocytes (hESC-CMs). In some embodiments, the cardiomyocytes are human induced pluripotent stem-cell derived cardiomyocytes (hiPSC-CMs). In some embodiments, the cardiomyocytes are human embryonic stem-cell derived cardiomyocytes (hESC-CMs).

In some embodiments, the ECT composition further comprises a hydrogel substantially comprised of collagen in a density of about 1 mg/ml to about 8 mg/mL. In some embodiments, the ECT composition further comprises a hydrogel substantially comprised of collagen hydrogel in a density of about 1 mg/mL to about 4.1 mg/mL. In some embodiments, the ECT composition further comprises a hydrogel substantially comprised of collagen in a density of about 3.5 mg/mL.

In some embodiments, the collagen hydrogel in the ECT composition is at a concentration of about 0.05 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/ml, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/ml, about 3.8 mg/mL, about 3.9 mg/mL, about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, or about 5.0 mg/mL.

Aspects of the present disclosure are directed to compositions that include cardiomyocytes at a density of about 5 to about 75 M/mL and a collagen hydrogel at a concentration of about 1 to about 3.5 mg/mL.

In some embodiments, the cardiomyocyte density is about 50 M/mL and the collagen hydrogel concentration is about 3.5 mg/mL. In some embodiments, the collagen hydrogel is derived from rat tail collagen.

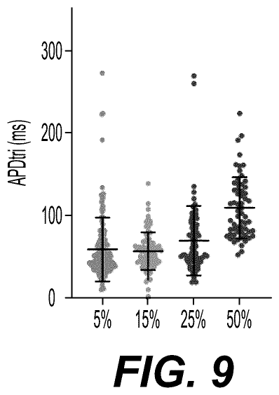

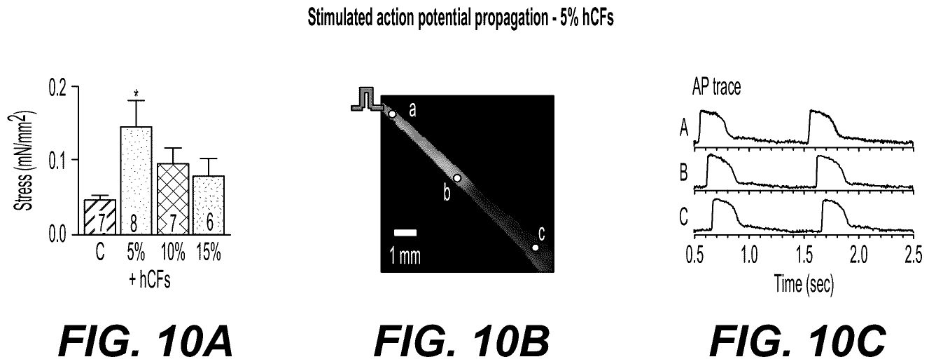

In some embodiments, the ECT composition further comprises cardiac fibroblasts (CFs) up to about 50%, or up to about 25% the number of CMs. In some embodiments, the number of CFs is about 5% to about 50% of the number of CMs. In some embodiments, the number of CFs is about 5% to about 25% of the number of CMs. In some embodiments, the number of CFs is about 5% to about 15% of the number of CMs. It was found that the amount of CFs can modulate the electromechanical function of ECTs. An amount of CFs that is too low leads to poor electromechanical function in the engineered tissue. A smaller population (e.g., about 5% of total cell number) of CFs improves ECT formation, material properties, and contractile function. When the amount of CFs is too high (e.g., 15% or more) the ECTs have increased ectopic activity and/or spontaneous excitation rate. Accordingly, in some embodiments, the number of CFs is about 5% of the number of CMs. In some embodiments, the CFs are human CFs. FIG. 9 shows that electrophysiology of ECTs at 5-25% CFs is normal but at 50% CFs, the electrophysiology is not normal. Action potential duration (APD) measures the length of the cardiac action potential (AP). APD triangulation (APDtri) is a measure of the slope of the decreasing portion. An increased APDtri is associated with increased arrhythmia risk, as it suggests HERG channel block. Thus, in FIG. 9, the stable and unchanging APDtri at lower CF % is an indicator of normal physiology and low arrhythmia risk, whereas an increasing APDtri suggests increasing arrhythmia risk. FIG. 10A shows fraction of beats in ECTs following 1 Hz stimulation for 5%, 10% and 15% CFs. FIG. 10B shows fluorescence image of paced beats from 5% hCF ECT, stimulated from the left upper corner. FIG. 10C shows sample action potential traces from the locations marked with “A,” “B,” and “C” in FIG. 10B. Thus 15% CFs may be an upper limit of concentration for CFs.

In some embodiments, the ECT composition has a volume that is surgically implantable in a living animal. In some embodiments, the ECT composition has a volume ranging from about 25 microliter (μL) to about 25 milliliter (mL). As used herein, a “surgically implantable” ECT or “surgically implantable volume” of an ECT composition refers to an ECT that can fit on the surface of the heart and/or does not cause pericardium effusion (buildup of extra fluid around the heart) and/or can be handled and sutured by a surgeon, and/or can be affixed/sutured within the space constraint of the thoracic cavity. In some embodiments, a surgically implantable ECT construct is suitable for covering at least a part of the heart of a subject.

In some embodiments, the composition further includes growth factors, cytokines or a combination thereof. In some embodiments, the growth factors include one or more of the following: insulin-like growth factor-1 (IGF-1), transforming growth factor-beta (TGF-β), vascular endothelial growth factor (VEGF), fibroblast growth factor-2 (FGF-2), hepatocyte growth factor (HGF) and platelet-derived growth factor (PDGF). In some embodiments, the cytokines include one or more of the following: interleukin-4 (IL-4), interleukin-6 (IL-6), interleukin-10 (IL-10), tumor necrosis factor-alpha (TNF-α), interferon-gamma (IFN-γ) and stem cell factors.

In some embodiments, the cardiomyocytes are derived from human induced pluripotent stem cells (hiPSC-CMs). In some embodiments, the hiPSC-CMs are derived from the WTC-11 GCaMP hiPSC line.

In another aspect, provided is a mold comprising the ECT composition described herein. It was found that the shape of the mold, and the placement of posts allows for optimization of tissue generation and minimizes tissue breaks. In some embodiments, the posts are rounded posts. In some embodiments, using rounded posts instead of posts with edges allows for optimization of tissue generation and minimizes tissue breaks. As used herein, “rounded posts” refers to rounded geometry with stabilizing posts to help with uniformity of stress during compaction and nutrient diffusion.

Also provided is a method for generating heart tissue in a subject, the method comprising implanting the ECT composition described herein in the subject in need thereof. In some embodiments, the subject suffers from cardiovascular disease (CVD). In some embodiments, the subject has had at least one episode of myocardial infarction (MI).

In some embodiments, the ECT composition described herein is used for generating cardiac tissue (e.g., after cardiac damage). In some embodiments, the ECT composition described herein is used for regenerating cardiac tissue (e.g., after cardiac damage). In some embodiments, the ECT composition described herein is used for improving cardiac tissue survival (e.g., after a myocardial infarction). In some embodiments, the ECT composition described herein is used for recovery of cardiac tissue function subsequent to cardiac tissue suffering damage. In some embodiments, the cardiac tissue survival/recovery is in vitro, e.g., the cultured ECT may have mechanical integrity and/or reduce ripping during manufacturing, i.e. the ECT is amenable to handling and transfers. In some embodiments, cardiac tissue survival is in vivo, i.e., the ECT can be grafted in the vicinity of a myocardium and the cells within the ECT divide and/or mature and/or differentiate in vivo to remuscularize the heart.

### Methods of Forming ECT

Provided is a method of forming an engineered cardiac tissue (ECT) construct suitable for covering at least part of the heart of a subject, the method comprising the steps of:
(a) providing a cell solution comprising human induced stem cell differentiated cardiomyocytes (hiPSC-CMs) and human fibroblasts wherein the number of human fibroblasts is about 5% to about 25% of the number of hiPSC-CMs;
(b) providing a hydrogel substantially comprised of collagen;
(c) preparing a casting mix by combining the cell solution of step (a) with the hydrogel of step (b) such that the hiPSC-CMs are dispersed throughout and suspended within the hydrogel;
(d) incubating the mixture of step (c) in a mold system having a defined shape to form an engineered tissue having a shape defined by the surface of the mold;
(e) culturing the engineered tissue up to about 7 days; and
(f) separating the tissue construct from the molding system to obtain the ECT construct suitable for covering at least part of the heart of a subject.

Provided is a method of forming an engineered cardiac tissue (ECT) construct suitable for covering at least part of the heart of a subject, the method comprising the steps of:
(a) providing a cell solution comprising human induced stem cell differentiated cardiomyocytes (hiPSC-CMs) and human fibroblasts;
(b) providing a hydrogel substantially comprised of collagen wherein the hydrogel has collagen in a density of about 3.5 mg/ml;
(c) preparing a casting mix by combining the cell solution of step (a) with the hydrogel of step (b) such that the hiPSC-CMs are dispersed throughout and suspended within the hydrogel;
(d) incubating the mixture of step (c) in a mold system having a defined shape to form an engineered tissue having a shape defined by the surface of the mold;
(e) culturing the engineered tissue up to about 7 days; and
(f) separating the tissue construct from the molding system to obtain the ECT construct suitable for covering at least part of the heart of a subject.

Provided is a method of forming an engineered cardiac tissue (ECT) construct suitable for covering at least part of the heart of a subject, the method comprising the steps of:
(a) providing a cell solution comprising human induced stem cell differentiated cardiomyocytes (hiPSC-CMs) and human fibroblasts;
(b) providing a hydrogel substantially comprised of collagen;
(c) preparing a casting mix by combining the cell solution of step (a) with a hydrogel of step (b) in a ratio ranging from about 1:1 to about 2:1 such that the hiPSC-CMs are dispersed throughout and suspended within the hydrogel;
(d) incubating the mixture of step (c) in a mold system having a defined shape to form an engineered tissue having a shape defined by the surface of the mold;
(e) culturing the engineered tissue up to about 7 days; and
(f) separating the tissue construct from the molding system to obtain the ECT construct suitable for covering at least part of the heart of a subject.

In some embodiments of the methods, the number of human fibroblasts in the cell solution is about 5% to about 15% of the number of hiPSC-CMs. In some embodiments of the methods, the number of human fibroblasts in the cell solution is about 5% of the number of hiPSC-CMs. In some embodiments of the methods, the number of human fibroblasts in the cell solution is about 10% of the number of hiPSC-CMs. In some embodiments of the methods, the number of human fibroblasts in the cell solution is about 15% of the number of hiPSC-CMs. In some embodiments, the human fibroblasts are primary human fibroblasts. In some embodiments, the human fibroblasts are differentiated human fibroblasts.

In some embodiments of the methods, the hydrogel has collagen in a density of about 1 mg/mL to about 8 mg/mL. In some embodiments of the methods, the hydrogel has collagen in a density of about 1 mg/mL to about 5 mg/mL. In some embodiments of the methods, the hydrogel has collagen in a density of about 1 mg/mL to about 4 mg/mL. In some embodiments of the methods, the hydrogel has collagen in a density of about 2 mg/mL to about 4 mg/mL. In some embodiments of the methods, the hydrogel has collagen in a density of about 3.5 mg/mL.

In some embodiments of the methods, the cell to gel ratio in the casting mix may be varied. In some embodiments, the casting mix is prepared by combining the cell solution of step (a) with a hydrogel of step (b) in a ratio ranging from about 2:1 to about 1: 2 such that the hiPSC-CMs are dispersed throughout and suspended within the hydrogel. In some embodiments of the methods, the casting mix is prepared by combining the cell solution of step (a) with a hydrogel of step (b) in a ratio of 1:1.

In some embodiments of the methods, the human fibroblasts are primary human fibroblasts. In some embodiments of the methods, the human fibroblasts are differentiated human fibroblasts. In some embodiments of the methods, the human fibroblasts are human cardiac fibroblasts. In some embodiments of the methods, the collagen is rat tail collagen. In some embodiments of the methods, the ECT construct obtained comprises up to about 1 billion cardiomyocytes (CMs) and has a CM density of about 5 million CMs/mL to about 75 million CMs/mL. In some embodiments of the methods, the ECT construct obtained comprises up to about 1 billion cardiomyocytes (CMs) and has a CM density of about 15 million CMs/mL, about 20 million CMs/mL, about 25 million CMs/mL, about 30 million CMs/mL, about 35 million CMs/mL, about 40 million CMs/mL, about 45 million CMs/mL, about 50 million CMs/mL, about 55 million CMs/mL, about 60 million CMs/mL, about 65 million CMs/mL, about 70 million CMs/mL, or about 75 million CMs/mL. In some embodiments of the methods, the ECT construct obtained comprises about 50 million CMs/mL.

In some embodiments of the methods, the engineered tissue is cultured for a period ranging from about 1 day to about 5 weeks. In some embodiments of the methods, the engineered tissue is cultured for a period ranging from about 1 day to about 30 days. In some embodiments of the methods, the engineered tissue is cultured for a period ranging from about 1 day to about 4 weeks. In some embodiments of the methods, the engineered tissue is cultured for a period ranging from about 1 day to about 3 weeks. In some embodiments of the methods, the engineered tissue is cultured for a period ranging from about 1 day to about 2 weeks. In some embodiments of the methods, the engineered tissue is cultured for a period ranging from about 1 week to about 5 weeks, about 4 weeks, about 3 weeks, or about 2 weeks. In some embodiments of the methods, the engineered tissue is cultured for a period of up to 10 days, up to 7 days, up to 5 days, up to 3 days or up to 1 day. In some embodiments of the methods, culturing the engineered tissue provides compacted engineered tissue. In some embodiments of the methods, the method provides improved mechanical integrity and/or reduced breakage of the engineered tissue.

In some embodiments of the methods, the ECT construct obtained reduces or eliminates the occurrence of arrythmias. In some embodiments of the methods, the ECT construct obtained does not exhibit spontaneous beating that is faster than the set pacing cycle length.

As shown in the Examples, the impact of certain biomanufacturing parameters including and not limited to cell dose, hydrogel composition, size of the formed ECT and function is evaluated. ECTs are fabricated by mixing human induced pluripotent stem-cell-derived cardiomyocytes (hiPSC-CMs) and human cardiac fibroblasts into a collagen hydrogel to engineer meso-(3×9 mm), macro-(8×12 mm), and mega-ECTs (65×75 mm). Meso-ECTs exhibited a hiPSC-CM dose-dependent response in structure and mechanics, with high-density ECTs displaying reduced elastic modulus, collagen organization, prestrain development, and active stress generation. Scaling up, cell-dense macro-ECTs are able to follow point stimulation pacing without arrhythmogenesis. Finally, a mega-ECT is successfully fabricated at clinical scale containing 1 billion hiPSC-CMs and implantated in a swine model of chronic myocardial ischemia to demonstrate the technical feasibility of biomanufacturing, surgical implantation, and engraftment.

### Examples

### Protocol: Engineering Clinically Sized Cardiac Tissue

Component 1: Human Induced Pluripotent Stem Cell Derived Cardiomyocytes (hiPSC-CMs)

hiPSC Maintenance. The WTC-11 GCaMP hiPSC line, is utilized for all experimental work. For maintenance culture, WTC-11s are seeded in 10 cm² dishes coated with 5 ug/mL vitronectin (VTN-N, Thermo Fisher, Waltham, MA) and maintained in Essential 8 (E8) medium (Gibco) within a cell culture incubator (37° ° C., 5% CO₂). When WTC-11s reach 80% confluency (4-5days), the cells are passaged using Versene (0.5 M EDTA, 1.1 mM D-glucose, Millipore Sigma) in Dubelco's Phosphate Buffered Saline (DPBS) without calcium and magnesium (Gibco).

Differentiation of hiPSC-CMs—Freezing/Thawing, Expansion and Selection. HiPSC are differentiated into cardiomyocytes using a well-adopted, chemically-defined small molecule protocol (pubmed.ncbi.nlm.nih.gov/23257984). Briefly, upon passage, hiPSCs are replated on 0.1 mg/mL Geltrex-coated (Gibco) 24-well plates in E8 medium with 10 μM Y-27632 (Rock Inhibitor, RI; Tocris, Bristol, UK). The next day, hiPSCs are treated with the Wnt activator CHIR 99021 (3-5 μM Chiron; Tocris, Bristol, UK) in CDM3 media for 24 h. Then, the Wnt inhibitor IWP2 (5 μM, Tocris, Bristol, UK) is added 72 h after CHIR 99021 treatment. The differentiating cells are fed with CDM3 every other day until day 9 when the media is changed to RPMI/B27 (Gibco).

At day 11, hiPSC-CMs are harvested and prepared for cryogenic storage to generate cell banks. For harvest, hiPSC-CMs culture plates are washed with DPBS followed by incubation in TrypLE™ Select Enzyme (TrypLE10×; Gibco). Cells are lightly triturated before transferring to a collection tube with an equal volume of RPMI/B27 supplemented with 10% Fetal Bovine Serum (FBS; Gibco) and 100 U/mL DNase I (DNase; Thermo Fisher Scientific, Waltham, MA, USA). Cells are counted with a hemocytometer and centrifuged (5 min, 300×g). The cell pellet is resuspended with CryoStor®CS10 (Stem Cell, Vancouver, Canada) at a concentration of 5-10 M/mL. Vials are placed inside a room-temperature CoolCell and frozen at −80° C.overnight. For long-term storage, cell vials are transferred to liquid nitrogen the following day.

For expansion, hiPSC-CMs from liquid nitrogen storage are thawed in a 37° C.water bath. The thawed cell solution is transferred directly to a large volume of RPMI/B27 and centrifuged (5 min, 300×g). The cell pellet is isolated and re-suspended in RPMI/B27+5 μM RI. Cells are seeded on a Geltrex-coated plate at low density (˜300,000 hiPSC-CMs/cm² plate) in either 15 cm plates or T225 flasks. Proliferation of terminally differentiated hiPSC-CMs is induced using low-concentration Wnt activation (2 μM Chiron), which increases cell counts by approximately 3-fold. Metabolic selection is then performed in glucose-free medium (DMEM (−) glucose) supplemented with 4 μM sodium lactate (MilliporeSigma, Cleveland, OH, USA), followed by Wnt inhibition with 4 μM Wnt-C59 (Tocris). This protocol yields cardiomyocyte purity >70% quantified by flow cytometry analysis of cardiac troponin T (cTnT) expression, with a range of 71.86-93.33% used in this study unless otherwise noted. All ECTs, including the mega-ECT containing 1 billion cardiomyocytes, are fabricated with hiPSC-CMs generated from this differentiation, expansion, and selection procedure.

Component 2: Primary Human Cardiac Fibroblasts (hCFs)

Cardiac Fibroblast Maintenance. Human cardiac fibroblasts (hCFs; Lonza) are maintained on 15 cm² plates with hCF media composed of DMEM/F12 (Gibco), 10% FBS, 4 ng/ml bFGF (Stemgent, Beltsville, MD, USA), and 100 μg/mL penicillin-streptomycin (penstrep; MilliporeSigma, Cleveland, OH, USA). Upon confluency, hCFs are passaged using 0.05% trypsin (Gibco) in Versene and frozen back in hCF media with 10% dimethyl sulfoxide (DMSO; Fisher Scientific, Waltham, MA, USA). All experiments use 5% hCFs between passages 5-7. HCFs are counted and viability assessed by trypan blue upon thawing before being used within ECTs.

### Component 3: High-Concentration Collagen Hydrogel

Isolation of collagen from rat tail. Rat tails are collected from Sprague-Dawley rats and stored at −80° C. for up to one year. For collagen isolation, tails are thawed at room temperature for 2 hours. The exterior of the tails is washed with isopropyl alcohol. Collagenous tendon fibers are isolated using the “twist and pull” method in which two pliers are used to grip and twist one end of the tail in order to isolate the tendon. This is performed along the total length of the tail. Isolated tendons are washed in PBS then transferred to 0.1M acetic acid in DI water at 4° C. with agitation (˜120 rpm stir bar) for ˜3 days to facilitate tendon degradation. After ˜3days, tissue remnants are removed from the solution through centrifugation (4700×g at 4 deg C. for 2 hours) to isolate the pellet. Slowly, 5M NaCl is added to the isolated pellet to a final concentration of 4% NaCl to promote collagen precipitation out of solution. After 1 hour, the solution is centrifuged again (4700×g at 4° C. for 2 hours), this time to isolate the pellet. The pellet is re-dissolved to 26 mg/mL (concentration determined via lyophilization) with 0.1M acetic acid.

Sterilization of high-concentration collagen from rat tail. The collagen extracted from rat tails is sterilized by floating the solution on top of chloroform (10% of collagen volume) in a bottle overnight at 4° C. The collagen solution is aseptically pipetted off the chloroform and stored at 4° C. for downstream application.

Working collagen hydrogel solution. All steps must be performed on ice to make the collagen working solution. For the clinical ECT, the stock concentration should be ×3 the desired final volume. The target is 3.5 mg/ml of collagen (final volume) so the working solution is 10.5 mg/mL. Combine 1:10 10× RPMI 1640 (Gibco; 31800089), 1:100 HEPES (Sigma; H0887-100ML), collagen (diluted from 26 mg/mL), 10M NaOH to pH 7-7.5 and mH₂O to achieve the final desired volume.
For example: 20 mL of 10.5 mg/mL collagen working solution
   2 mL 10× RPMI
   200 μL HEPES
   60 μL 10M NaOH
   9.74 mL mH₂O
   8 mL collagen (26 mg/mL stock)

### Component 4: Mold Design

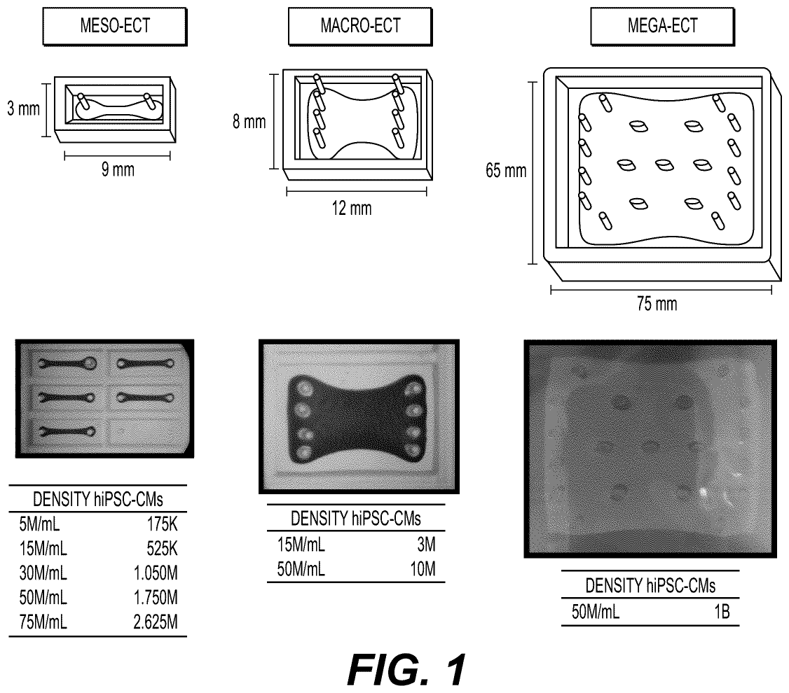

Molding System and Modification for Scale Up. Polydimethylsiloxane (PDMS) molds for ECT formation and culture are created using a simple replica-molding workflow. For this study, three molding systems are used for iterative, progressive scale up: (1) meso-molds: 3×9 mm, 35 μL; (2) macro-molds: 8×12 mm, 200 μL; (3) mega-molds: 65×75 mm, 20 mL (FIG. 1). Negative acrylic mold fabrication. Negative templates are laser-etched in ½-inch acrylic sheets. For laser etching, a Universal Laser Systems 6.75 Laser Cutter (ULS) at the Brown Design Workshop (Brown University) is utilized to create such acrylic templates.

PDMS mold fabrication and preparation for culture. Stylgard 184 (PDMS; Dow) is then cast on the acrylic templates and cured overnight at 60° C. The cured PDMS molds are removed from the acrylic templates and sterilized with an autoclave at 121° C. for 30 minutes. If tissue is sticking, one hour immediately prior to casting, Pluronic (10% in milliQ-H₂O; Sigma) can be used to coat the PDMS trough to facilitate cell spreading and prevent wall attachment.

### Combining Components

### Cell to Gel Ratio of 2:1

To fabricate the clinical ECT, a hiPSC-CM cell solution and collagen hydrogel working solution is combined at a volumetric ratio of 2:1. The total volume used for the clinical ECT mold is 21 mL—this means 14 mL of cell solution (75 M/mL) and 7 mL of collagen hydrogel working solution (10.5 mg/mL). The final concentration of cells is 50 M/mL and collagen will be 3.5 mg/mL.

Harvest hiPSC-CMs according to the protocol above. Harvest hCFs according to the protocol above. Combine hiPSC-CMs with 5% hCFs. (for example: 1 billion hiPSC-CMs+50 million hCFs=1.05 billion cells). Re-suspend at a cell concentration of 75 M/mL. Degas the collagen working solution on ice. Immediately after, add the collagen working solution to the cell solution with the appropriate volume. For example, for 1 tissue system, if there is 14 mL of cells then add 7 mL of collagen working solution). Pipette 21mL of the cell and collagen mixture to the PDMS mold. Incubate in 37° C. for 30-45 minutes and check periodically for proper gelling. Feed the tissue with metabolic maturation media (MM) (described below)+5 μM RI. Replace/replate media 24 hours later with fresh metabolic maturation media. Monitor in culture for 3-7 days with daily media changes.

### Cell to Gel Ratio of 1:1

Fabrication of Engineered Cardiac Tissue (ECTs). To fabricate ECTs, hiPSC-CMs are harvested using TrypLE10× and combined with 5% hCFs from thawing. The cell solution is diluted in RPMI/B27 media to the desired density and combined 1:1 with a collagen hydrogel solution. Two collagen sources are used with different stock concentrations to enable increasing the collagen concentration (from 1 to 3.5 mg/mL) when scaling up from meso-ECTs (1 mg/ml collagen concentration unless otherwise noted) to macro-ECTs (3.5 mg/mL collagen concentration) in order to balance the increased cell densities and tissue size. A commercial stock of rat tail type I collagen (3.9-4.1 mg/ml; Advanced BioMatrix, Carlsbad, CA, USA) is utilized for the generation of all 1 mg/mL collagen hydrogel ECTs. The hydrogel casting mix is prepared by combining the collagen stock with 10× RPMI 1640 (1:10; Gibco), HEPES (1:100; MilliporeSigma, Cleveland, OH, USA), mH₂O and finally neutralizing the mix with 5 M sodium hydroxide to a pH between 7 and 7.5. To achieve the collagen concentration of 3.5 mg/mL, however, a higher stock concentration of type 1 collagen is required. Therefore, collagen type 1 is isolated in-house from rat tails. Briefly, rat tails are collected from Sprague-Dawley rats and stored at −80° C. for up to one year. For collagen isolation, tails are thawed at room temperature for 2 h. The exterior of the tails is washed with isopropyl alcohol, and collagenous fibers are harvested by twisting the tail to expose the tendon. Isolated tendons are washed in PBS and then transferred to 0.1 M acetic acid in mH₂O at 4° C. with agitation (˜120 rpm stir bar) for ˜3 days to facilitate tendon degradation. After ˜3 days, tissue remnants are removed from the solution through centrifugation (4700×g at 4° C. for 2 h). To the supernatant, 5 M sodium chloride (NaCl; MilliporeSigma, Cleveland, OH, USA) in water is added to a final concentration of 4% NaCl to promote collagen precipitation out of solution. After 1 h, the solution is centrifuged again (4700×g at 4° C. for 2 h), this time to isolate the pellet. The pellet is reconstituted at 26 mg/mL (concentration determined via lyophilization) in 0.1 M acetic acid. The collagen extracted from rat tails is sterilized by floating the solution on top of chloroform (10% w/v; MillporeSigma, Cleveland, OH, USA) in a bottle overnight at 4° C. The collagen solution is then aseptically pipetted off the chloroform and stored at 4° ° C. for downstream applications.

Once combined, the cell-hydrogel casting mix is immediately pipetted into the desired PDMS mold system and allowed to gel at 37° C. for 30-45 min. After which, the ECTs are cultured in a metabolic maturation medium (MM), composed of DMEM without glucose (Gibco) supplemented with 3 mM glucose (MilliporeSigma, Cleveland, OH, USA), 10 mM L-lactate (MilliporeSigma, Cleveland, OH, USA), 5 mg/mL Vitamin B12 (MilliporeSigma, Cleveland, OH, USA), 0.82 mM biotin (Millipore-Sigma, Cleveland, OH, USA), 5 mM creatine monohydrate (MilliporeSigma, Cleveland, OH, USA), 2 mM taurine (MilliporeSigma, Cleveland, OH, USA), 2 mM L-carnitine (MilliporeSigma, Cleveland, OH, USA), 0.5 mM ascorbic acid (MilliporeSigma, Cleveland, OH, USA), 1×NEAA (Gibco), 0.5% (w/v) Albumax (Thermo Fisher Scientific, Waltham, MA, USA), 1×B27, and 1% knockout serum replacement (KOSR; Gibco). ECTs are cultured at 37° C., 5% CO2, and stimulated with a 4 ms biphasic field pulse stimulus at 1 Hz and 4 V/cm using a 6-well electrode insert (C-Dish, IonOptix, Westwood, MA, USA) connected to the IonOptix culture pacing system (C-Pace EP, IonOptix, Westwood, MA, USA) for up to 7 days prior to analysis.

ECT Survival, Compaction and Pacing Analysis during Culture. Nondestructive, longitudinal brightfield optical microscopy images and videos (Olympus SZ40) are taken of ECTs daily. Survival curves are generated by monitoring tissue state throughout culture, with intact ECTs being defined as having no breakage either internally or at the PDMS posts. Survival curves and percent survival are calculated by plotting the number of intact tissues (as total minus broken tissues) over the 7 days of in vitro culture with Prism 8 (GraphPad). Using the daily images, ECT compaction is calculated by measuring the two-dimensional (2D) tissue area for each ECT in ImageJ. Two-dimensional areas are normalized to the area of the ECT on day 0 to calculate the fraction of 2D tissue area needed to assess the extent of compaction. Daily videos of each ECT beating in culture are used to count the number of contractile beats, which is divided by the time analyzed to calculate beats per minute (BPM).

Mechanical Analysis. The passive (Young's modulus) and active (contractile stress generation) mechanical properties of the meso-ECTs are analyzed using a custom micromechanical tensile apparatus (Aurora Scientific, Aurora, Canada). After 7 days in 3D culture, meso-ECTs are mounted on the tensile apparatus in a 37° C. bath of Tyrode's solution. Tissues are stretched to 130% of their initial length in 5% increments and held for 120 s at each strain to allow for stress relaxation. During the last 20 s of this hold, an electrical stimulus of 1 Hz is administered to determine active stress contraction at 5% strain intervals up to 30%. At 30% stretch, the force-frequency response of the tissue is determined by increasing the electrical stimulus from 1 Hz to 4 Hz in 0.5 Hz intervals. A custom MATLAB (Mathworks, Natick, MA, USA) script is utilized to analyze active contractile amplitude (force, mN; active stress generation, mN/mm²) and kinetics (upstroke velocity, mN/mm²/s; time to 50% relaxation and 90% relaxation, ms). Contractile alternans are also quantified, defined as the change observed in contractile amplitude. The force-frequency response is recorded, with the maximum capture rate (MCR) as the maximum frequency at which the ECT could follow the target pacing. Meso-ECTs, as well as macro- and mega-ECTs that can not be securely mounted to the tensile testing apparatus, are analyzed using the video-based analysis software MUSCLEMOTION® to determine contractile amplitude.

Curling Angle and Prestrain Measurement. Curling angle and prestrain calculations are made as previously described. After 7 days of in vitro culture, meso-ECTs being mechanically tested are removed from their PDMS posts, which anchor the tissue during culture. The meso-ECTs are transferred to a well plate with fresh media and allowed to sit at 37° C. for 10 min, defined as a “stress-free” environment in which the tissue can fully release any residual stress. Brightfield images are taken of the tissue to assess the different recoil profiles of density conditions. ImageJ (NEI, Bethesda, MD) is then used to quantify the curling angle of this tissue, which is defined as the angle made between the tissue endpoints and midpoints. To estimate pre-strain in 1D, tissue displacement (u) is calculated by assuming the initial tissue position is fully linear (x, curling angle=180°). ImageJ is then used to acquire x and y coordinates. From this displacement, the deformation tensor (1) and green strain (2) can be calculated.

F(x)=Δu(x)/Δx (1)

E(x)=0.5F(x) (2)

Immunohistochemical Staining, Imaging and Analysis of ECTs. ECTs are fixed in 4% paraformaldehyde (MilliporeSigma, Cleveland, OH, USA) and washed with DPBS. For short-term storage, samples are kept at 4° C. For staining, ECT samples are frozen and embedded in optical cutting temperature medium or processed in paraffin, followed by sectioning at 5 μm. Unless otherwise noted, ECTs are sectioned lengthwise through the full thickness of the tissue. Multiple sections throughout the ECT thickness (≥3) are used for staining. To visualize collagen content, picrosirius red/fast green (PRFG) staining is performed on sections, followed by imaging using an Olympus FV200 Slide Scanner. Following this imaging, second harmonic generation (SHG) imaging is performed on an Olympus FV1000 MPE Multiphoton Microscope to examine collagen organization.

For immunohistochemical staining, sectioned samples are blocked with 1% normal goat serum (NGS; MilliporeSigma, Cleveland, OH, USA) in DPBS for 1 h followed by incubation with primary antibodies overnight at 4° C. The following day, sections are incubated with secondary antibodies and nuclear counterstains for 1 h at room temperature. Primary and secondary antibodies are listed in Table 1. Coverslips are mounted using Prolong AntiFade Glass Mountant (Invitrogen, Waltham, MA, USA). Once set, sections are imaged using an Olympus FV3000 confocal microscope and processed in ImageJ. Quantification of percent area is performed for all PRFG, SHG, and immunohistochemical targets. First, images are separated by channel, thresholded, and binarized. In the case of PRFG and SHG images, the percent area of the stain is normalized by the area of tissue analyzed, while sarcomeric α-actinin and wheat germ agglutinin (WGA) content is normalized by the number of nuclei. In conditions where sarcomeres are clearly visible, sarcomere length is determined by measuring the distance between 3-5 sarcomeres in a straight line and dividing by the number of sarcomeres. For quantification of content for the meso- and macro-ECTs, multiple (3-6) regions are averaged per ECT analyzed. For quantification of nuclei size in the meso-ECT and macro-ECTs, nuclei from multiple (3-6) regions are averaged (minimum 50 nuclei total) per ECT analyzed (represented by individual points on the graphs). For the mega-ECT, in which one tissue is analyzed, the quantification of content and nuclei is averaged per area analyzed (6 regions, represented by individual points on the graph).

TABLE 1
Antibodies used in immunohistochemical staining

| Primary/Secondary Antibody | Dilution | Catalog Number |
|---|---|---|
| Mouse monoclonal anti- | 1:500 | MilliporeSigma; A7811- |
| α-sarcomeric actinin | | 0.2ML |
| Mouse monoclonal anti-cTnT | 1:100 | Invitrogen; MA5-12960UL |
| Rabbit polyclonal anti-MLC2v | 1:100 | ProteinTech; 10906-1-AP |
| Mouse monoclonal anti-MLC2a | 1:100 | Synaptic Sytems; 311 011 |
| Bisbenzimide H 33342 | 1.5 μg/mL | MilliporeSigma; B2261- |
| trihydrochloride (Hoechst) | | 100MG |
| Goat anti-mouse/rabbit | 1:250 | Invitrogen; A1100/A11008 |
| Alexa Fluor 488 | | |
| Goat anti-mouse/rabbit | 1:250 | Invitrogen; A11012/ |
| Alexa Fluor 594 | | A11005 |
| Wheat Germ agglutinin | 1:100 | CellSignalling Tech; |
| (WGA) Alexa Fluor 488 | | 13116S |

Optical Mapping of Calcium and Voltage Transients. To assess action potential (AP) and calcium kinetics in ECTs, a custom optical mapping system is utilized. Briefly, ECTs are stained with the voltage-sensitive dye, di-4-ANEPPS (Invitrogen, Waltham, MA, USA) and imaged in a 37° C. temperature-controlled bath perfused with a solution containing 140 mM NaC1, 5.1 mM KCl, 1 MgCl₂, 1 mM CaCl₂, 0.33 mM NaH₂PO₄, 5 mM HEPES, and 7.5 mM glucose; gassed with 95% O₂ and 5% CO₂; and supplemented with 5 μM blebbistatin, an acto-myosin inhibitor to reduce motion artifacts. ECTs are paced by a concentric bipolar stimulation electrode that has a 1.3 mm diameter (small stimulation electrode set for mouse hearts, Harvard Apparatus). The stimulation strength is set to 0.3 mA for a duration of 2 ms. Fluorescent images of calcium tracing (using the intrinsic GCaMP signaling) and Aps are simultaneously recorded using dual CMOS cameras (100×100 pixels, 1000 frames/s, 10×10 mm FOV; Ultima-L, SciMedia, Costa Mesa, CA, USA). Calcium and voltage activation maps are generated from the maximum time delay between the cross-correlation of each pixel versus a reference pixel (±300 ms). The conduction velocities are calculated as the slopes of the line of best fit through distance versus activation time along the line.

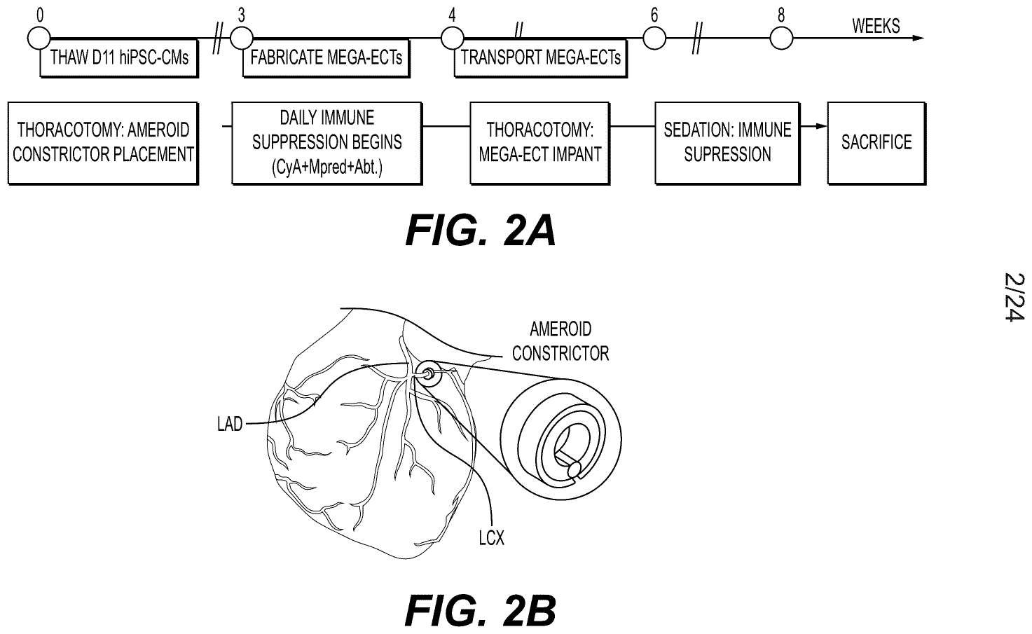

Animal Methods. All animal procedures are approved under IACUC #5024-21 at Rhode Island Hospital, according to the timeline in FIG. 2A. Two Yorkshire swine (male and female; Tufts University, Medford, MA, USA), aged 8 to 10 weeks and weighing between 22 and 25 kg at the time of the initial procedure, are used. This pilot study is designed to assess technical feasibility using an established model of chronic myocardial ischemia. To induce chronic ischemia, a small left thoracotomy is performed, the pericardium is opened, and an ameroid constrictor composed of hygroscopic casein material cased in titanium (Research Instruments SW, Escondido, CA, USA) is sized and placed around the proximal left circumflex (LCX) coronary artery (FIG. 2B). Slow swelling of the constrictor reduces blood flow, causing a slow onset of ischemia. Four weeks later, a second left thoracotomy is performed for implantation of the mega-ECT, which is secured to the epicardial surface with 4 sutures (6-0 prolene) at each corner of the mega-ECT. Heart rate and ECG activity are monitored daily throughout the study using a heart rate monitor belt (polar heart rate monitor chest strap). The terminal endpoint is four weeks after the implantation surgery to assess the engraftment and impact of hiPSC-CMs.

In order to promote engraftment and minimize xenograft rejection, an aggressive immune suppression protocol using cyclosporin A, methylprednisolone, and abatacept CTLA4 immunoglobulin is implemented. Trough measurements are taken throughout the study to monitor cyclosporin levels through a vascular access port (VAP) in the jugular vein that is implanted during the second surgery.

Statistical Analysis. Student t-tests as well as one-way or two-way repeated-measures analysis of variance (ANOVA) are used in statistical testing as appropriate. When necessary, the Tukey-Kramer method of post hoc analysis is performed, with p-values <0.05 considered statistically significant. All analysis is performed in Prism 8 (GraphPad) and reported with the standard error of the mean.

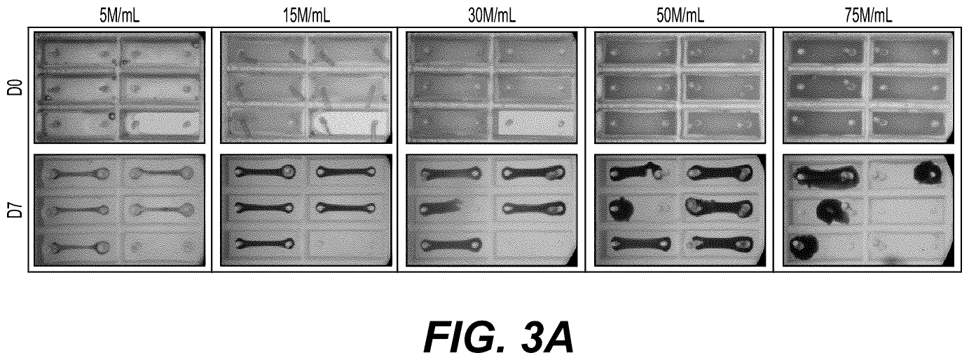

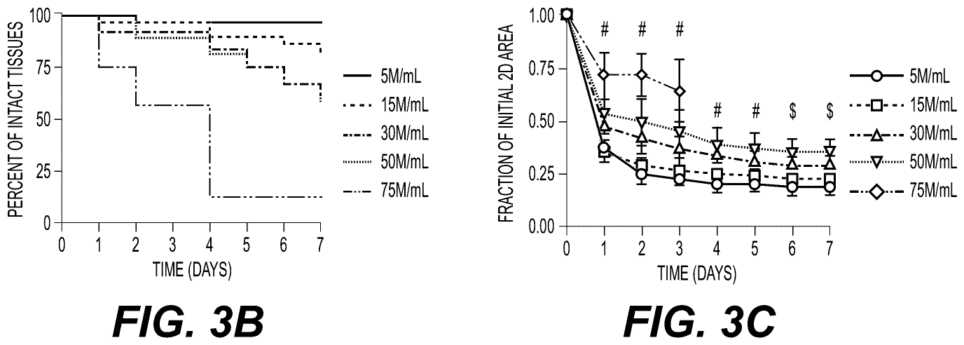

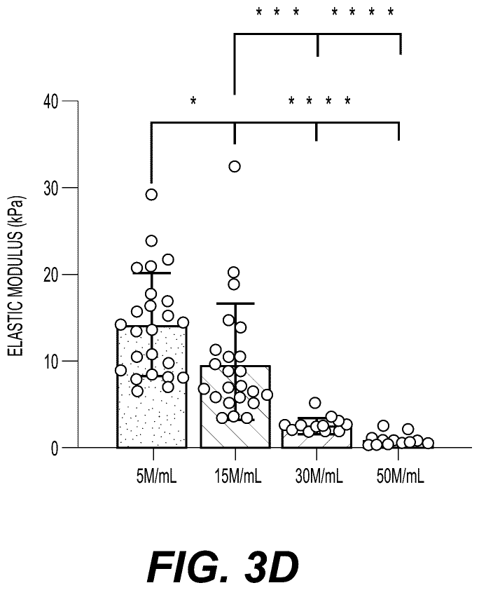

Cell Density Impacts Meso-ECT Syncytium Formation, Structural Integrity and Collagen Remodeling. To develop ECTs with therapeutically relevant hiPSC-CM numbers, tissue formation and function are studied across a range of cell densities (5 M/mL, 15 M/mL, 30 M/mL, 50 M/mL, and 75 M/mL) using the standard collagen hydrogel concentration of 1 mg/ml within all the meso-ECTs, unless otherwise noted. From brightfield images of the meso-ECTs over the 1-week culture period (FIG. 3A), it is evident that increasing cell density impacts tissue formation. Notably, increasing cell density decreases meso-ECT structural survival in culture (FIG. 3B), with cell-dense meso-ECTs displaying greater breakage. At just 2 days in culture, structural survival of 75 M/mL meso-ECTs is only 56.25%, indicating that the initial stages of tissue formation and compaction are severely compromised with high hiPSC-CM input. After 7 days in culture, all but the highest density of 75 M/mL. has >50% surviving intact tissues (5 M/mL: 96.15%; 15 M/mL: 82.76%; 30 M/mL: 62.50%; 50 M/mL: 59.26%; 75 M/mL: 12.50%). In culture, cell-dense tissues (50 M/mL and 75 M/mL) break primarily due to the appearance of small tears in the meso-ECT interior. In contrast, hypercompaction and necking is the predominant failure mode in the lower cell density conditions (5 M/mL, 15 M/mL, and 30 M/mL).

Over the 1-week culture period, the meso-ECTs compact to varying degrees, a process in which the embedded cells form adhesions to one another and the protein matrix, generates intracellular and tissue-level tension, as well as produce and remodel the surrounding matrix. Increased compaction is observed throughout the culture in the 5 M/mL and 15 M/mL conditions compared to 30 M/mL, 50 M/mL, and 75 M/mL at every time point (FIG. 3C). For all conditions, the majority of meso-ECT compaction occurs during the first 24 h of tissue formation. By day 4, compaction stabilizes, as defined by no changes >5% (5 M/mL: 0.20±0.01, 15 M/mL: 0.24±0.01, 30 M/mL: 0.32±0.01, 50 M/mL: 0.40+0.02, fractional area). Comparison of input cell density with calculated cell density after compaction (day 7) is also quantified (Table 2). Due to the particularly poor compaction and structural failure of 75 M/mL meso-ECTs, this group is excluded from further analysis.

TABLE 2
Density of Meso-ECTs input versus compacted

| Input Density | Calculated Volume | Density after Compaction |
|---|---|---|
| (hiPSC-CM/mL) | (D 7, mm³) | (hiPSC-CM/mL) |
| 5M/mL | 0.55 ± 0.06 | 318.18M ± 64.,28M |
| 15M/mL | 1.045 ± 0.06 | 502.39M ± 53.03M |
| 30M/mL | 1.98 ± 0.11 | 530.3M ± 59.12M |
| 50M/mL | 3.91 ± 0.28 | 448.14M ± 63.43M |

The structural integrity of meso-ECTs is next quantified by analyzing the elastic modulus, which shows an inverse relationship to cell density (5 M/mL: 14.09±1.22 kPa; 15 M/mL: 9.71±1.41 kPa; 30 M/mL: 2.46±0.25 kPa, 50 M/mL: 0.67±0.17 kPa) (FIG. 3D). These values are lower than those of the native myocardium (30.80±2.71 kPa in the longitudinal direction and 16.58±1.85 kPa in the transverse direction), yet greater than the acellular 1 mg/ml collagen hydrogel controls (0.28±0.03 kPa, mechanically tested after 24 h), suggesting increased stiffness with the addition of cells to the hydrogel, regardless of density. This inverse relationship between elastic modulus and cell density suggests that the low stiffness of the cell-dense meso-ECTs contributes to breakage, likely due to an imbalance of the cell: ECM ratio and physical space constraints (namely, cellular crowding) compromising adhesion formation and compaction that characterize tissue formation in vitro.

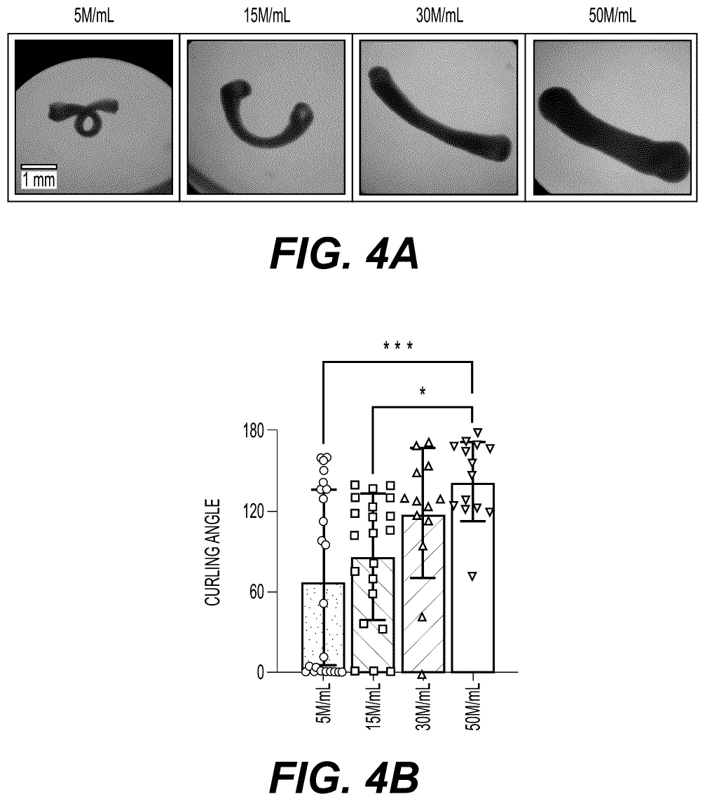

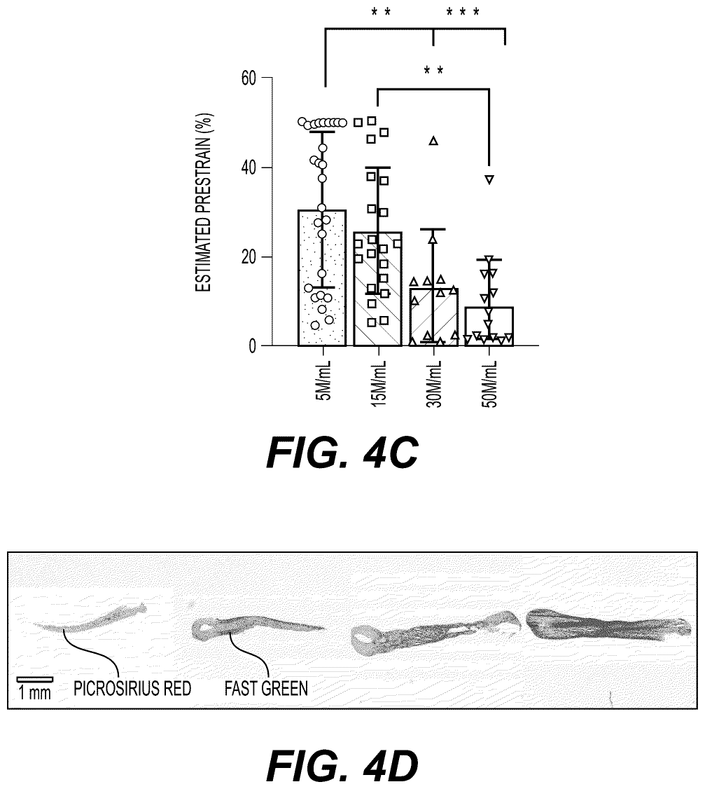

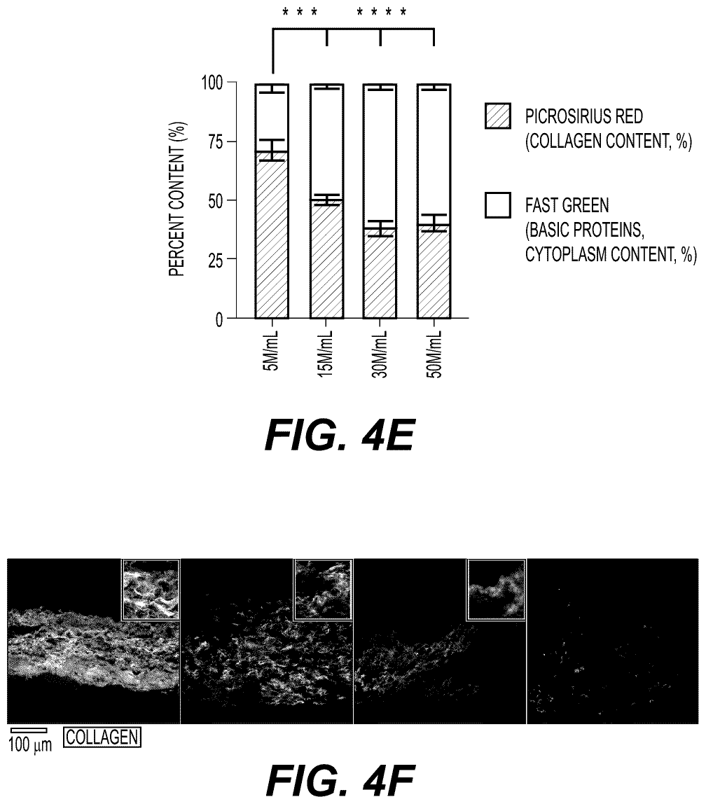

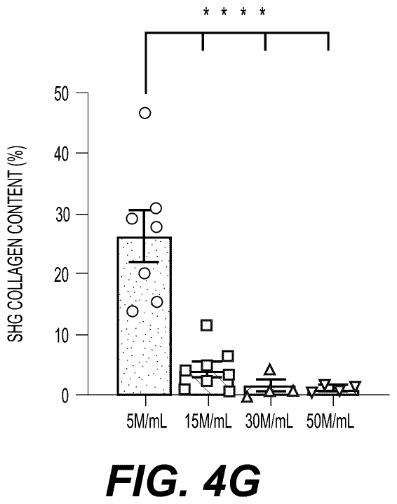

Although reduced elastic modulus can be partially explained by decreased compaction of the cell-dense meso-ECTs, the differences in the remodeled tissue environment, which may also contribute to these mechanics, need to be better understood. At the global tissue level, a different recoil profile of the meso-ECTs in a stress-free environment is observed (FIG. 4A). Decreased meso-ECT curling angles are measured at higher cell densities (5 M/mL: 67.01±14.19°; 15 M/mL: 85.95±10.44°; 30 M/mL: 118.0±13.59°; 50 M/mL: 141.6±7.99°) (FIG. 4B). As a corollary, prestrain, calculated from the tissue displacement in the stress-free environment, also shows that the highest cell densities have the lowest developed prestrain (5 M/mL: 30.68±3.60%; 15 M/mL: 25.67±3.12%; 30 M/mL: 13.32±3.63%; 50 M/mL: 9.13±2.71%) (FIG. 4C).

In addition, the cellular scale is looked at by quantifying PRFG staining of the meso-ECTs (FIG. 4D). The intensity of fast green stain (basic protein content, e.g., of the cytoplasm) shows an expected positive correlation with input density (5 M/mL: 28.80±4.32%; 15 M/mL: 49.45±2.29%; 30 M/mL: 61.59+3.15%; 50 M/mL: 59.77+3.33%, % area analyzed) (FIG. 4E). Quantification of picrosirius red (collagen content), on the other hand, shows a negative correlation with input density, as the highest collagen content appears in the lowest density condition (5 M/mL: 71.20±4.32%; 15 M/mL: 50.55±2.29%; 30 M/mL: 38.41+3.15%; 50 M/mL: 40.23±3.33%, % area analyzed). Recognizing that not only the amount of collagen present but also its organization may be important in its contribution to the structural and mechanical integrity of the tissues, collagen organization is quantified using SHG imaging, which is an imaging modality that shows fibrillar collagen which is either deposited by the cells within the ECT and/or reorganized by the cells within the ECT (as opposed to the collagen provided through the hydrogel) (FIG. 4F). Collagen present in the meso-ECTs with lower cell densities (5 M/mL and 15 M/mL) is highly organized, as demonstrated by the bright signal. Similar to the PRFG results, the results of collagen quantification from SHG reveal the highest amount of organized collagen in the lowest cell density conditions (5 M/mL: 26.27±4.26%; 15 M/mL: 4.08+1.24%; 30 M/mL: 1.49±0.93%; 50 M/mL: 0.59±0.27%, % area analyzed) (FIG. 4G). No collagen abnormalities characteristic of fibrosis are observed in any condition. An average ratio of the organized collagen content quantified by SHG normalized to the total general collagen content quantified through PRFG staining for each condition is calculated (5 M/mL: 36.89; 15 M/mL: 8.06; 30 M/mL: 3.88; 50 M/mL: 1.47, measured as SHG normalized to PRFG collagen content), illustrating a negative correlation between the organized-to-unorganized collagen ratio and increasing cell density. These results suggest that the organization of the ECM within the ECT is essential for the structural integrity of the tissue.

To confirm that the observed changes in collagen content and organization are due to active cell remodeling as opposed to the persistence or development of the initial collagen hydrogel, PRFG and SHG imaging are performed on acellular collagen constructs. PRFG staining of the acellular constructs illustrated dissimilar collagen content and structure, supporting the hypothesis that active cell remodeling of the hydrogel is occurring within the low-density meso-ECTs. Although SHG imaging is performed, no signal is obtained, suggesting the building of fibrillar collagen in the ECM occurs in ECTs during culture by the embedded cells as opposed to cell-initiated degradation. Additionally, because collagen deposition and organization can be influenced by the fibroblast-like non-myocytes arising from hiPSC-CM differentiation, the PRFG and SHG quantification are normalized by differentiation purity by multiplying the differentiation purity (cTnT+ as assessed by flow cytometry) and the quantification. Through this normalization, the raw quantification values slightly decrease, with greater convergence between cardiac differentiation batches observed and statistical significance maintained.

Together, these results show that increased hiPSC-CM density in meso-ECTs negatively affects the ability of the embedded cells to remodel, compact, and develop prestrain within their environment, which compromises the passive mechanical properties of the tissue and increases its risk for structural failure. While there exists a threshold required for tissue integrity (to avoid structural failure), a spectrum of tissue compositions with varying stiffnesses and fibrillar collagen content is likely to be acceptable for implementation as a regenerative therapy.

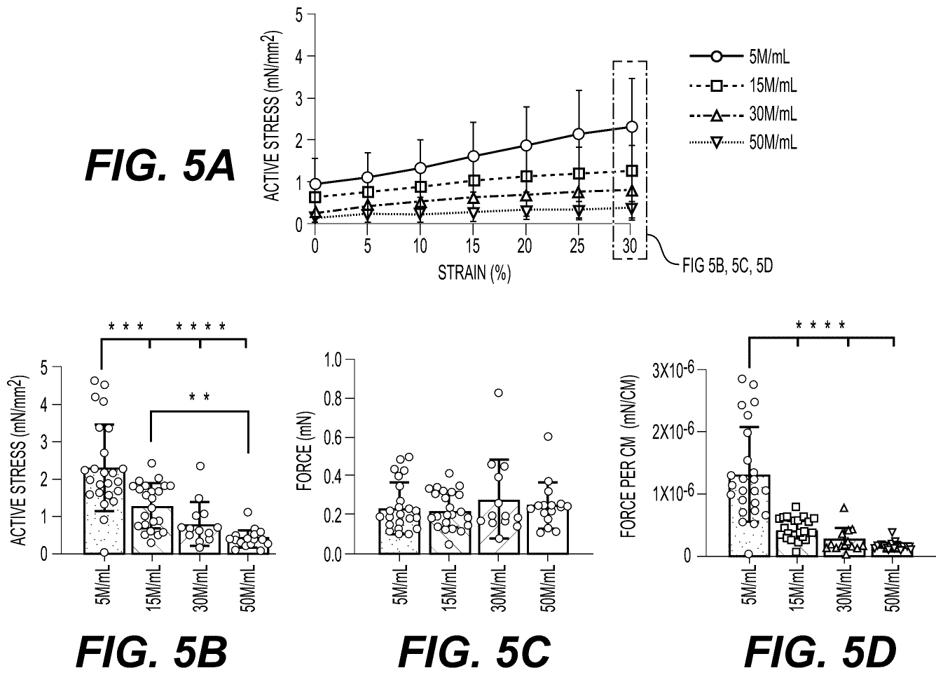

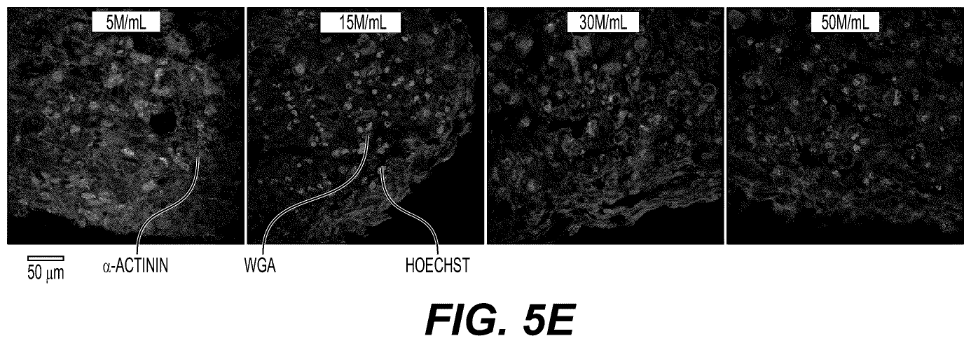

Physiological Analysis Reveals Cell-Dose Dependent Effects on Meso-ECT Contractile Stress and Sarcomere Structure. To understand the functional consequences of increasing hiPSC-CM density, the contractility of meso-ECTs is measured by uniaxial tensile testing. Lower cell density meso-ECTs exhibit increased active stress generation at all tested strains (0-30% strain; see e.g., FIG. 5A). At the highest strain of 30%, active stress generation is significantly higher in the 5 M/mL condition (2.32±0.24 mN/mm²) compared to both 15 M/mL and 30 M/mL conditions (15 M/mL: 1.29±0.12 mN/mm² and 30 M/mL: 0.79±0.16 mN/mm²), as well as the higher 50 M/mL density condition (50 M/mL: 0.38±0.06 mN/mm²) (FIG. 5B). At day 7, the cross-sectional area (CSA) of the meso-ECTs shows increasing CSA correlating with cell density (5 M/mL: 0.10±0.01 mm²; 15 M/mL: 0.19±0.01 mm²; 30 M/mL: 0.36±0.02 mm²; 50 M/mL: 0.71±0.05 mm²). Due to significant differences in ECT compaction, CSA is used to normalize force for stress analysis. Raw force and force normalized by the number of input hiPSC-CMs are also assessed. Interestingly, raw force generation is not statistically different between conditions, despite the increased number of hiPSC-CMs within the high-density meso-ECTs (FIG. 5C). Normalization of force by hiPSC-CM input further reveals a significant increase in force generation per hiPSC-CM in the low cell density conditions (5 M/mL: 1.31×10⁻⁶±0.156×10⁻⁶ mN/hiPSC-CMs) compared to the higher cell density conditions (15 M/mL: 0.43×10⁻⁶±0.04×10⁻⁶ mN/hiPSC-CMs; 30 M/mL: 0.27×10⁻⁶±0.05×10⁻⁶ mN/hiPSC-CMs; 50 M/mL: 0.14×10⁻⁶±0.02×10⁻⁶ mN/hiPSC-CMs) (FIG. 5D). In addition to contractile stress magnitude, contractile kinetics as measured by upstroke velocity (Vup) and time to 50% and 90% relaxation (T50 and T90, respectively) are also quantified. Vup is unchanged between density conditions, while small yet significant differences are detected in the time to relaxation when comparing 5 M/mL and 50 M/mL meso-ECTs across 10-25% strain, as expected from the higher force per cell in 5 M/mL conditions.

Because tensile mechanical analysis could only be performed on intact meso-ECTs at day 7, a video-based analysis of contractility using the software MUSCLEMOTION® is implemented. This method enables serial measurements for quantification of contractility throughout the culture to determine if there is a survival bias within the higher density conditions. Interestingly, the contractile amplitude measured through MUSCLEMOTION® analysis of the meso-ECTs on day 7 in culture and force generation at 0% stretch measured by uniaxial tensile testing does not correlate well (R²=0.001). Additionally, it is observed that contraction amplitudes, as measured through MUSCLEMOTION®, have a positive correlation with increasing cell density. This may be due to the different contraction types observed in the meso-ECT density conditions during culture, with lower density conditions contracting isometrically, thus maximizing force generation (uniaxial tensile stress) and limiting displacement (contraction amplitude measured through MUSCLEMOTION® 2D displacement), while higher density conditions contract isotonically, maximizing displacement.

In addition to analyzing the impact of cell density on the contractile properties of the meso-ECTs, the pacing frequency dependence of contraction is assessed, reporting a negative relationship for all meso-ECT conditions. Most tissues are able to be paced up to 4 Hz, although the average pacing of the low 5 M/mL cell density condition averaged 3.5 Hz. Additionally, meso-ECTs, irrespective of density condition, begin experiencing contractile alternans (defined as a >10% difference in beat-to-beat contractile amplitude) at 2.5 Hz pacing, which increases in magnitude with pacing as previously observed.

Histological staining is performed next to assess myofilament development and alignment (see e.g., FIG. 5E). These results illustrate that sarcomere length is not significantly different between conditions (5 M/mL: 1.29±0.03 μm; 15 M/mL: 1.27±0.03 μm; 30 M/mL: 1.16±0.04 μm; 50 M/mL: 1.24±0.05 μm). However, lower-density meso-ECTs (5 M/mL and 15 M/mL) display aligned sarcomeres throughout the tissue interior, while high-density meso-ECTs (30 M/mL and 50 M/mL) have limited sarcomere alignment only at the tissue edges. Further quantification of histological stains shows increased nuclear size of 5 M/mL meso-ECTs compared to 15 M/mL and 30 M/mL conditions (5 M/mL: 29.73±1.29 μm2;15 M/mL: 23.99±0.67 μm²; 30 M/mL: 24.20±0.78 μm²; 50 M/mL: 25.62±1.57 μm²). It is important to note that nuclear fragmentation, which is a hallmark of cells undergoing apoptosis and necrosis, is not found through the thickness of the meso-ECTs, suggesting that cellular viability is maintained. WGA and cfractinin content normalized by the number of nuclei is calculated, but no significant differences are found.

In addition to analyzing the impact of cell density on the contractile properties of the meso-ECTs, the pacing frequency dependence of contraction is analyzed. Most tissues were able to be paced up to 4 Hz, although the average pacing of the low 5 M/mL cell density condition averaged 3.5 Hz. Additionally, meso-ECTs, irrespective of density condition, began experiencing contractile alternans (defined as a >10% difference in beat-to-beat con-tractile amplitude) at 2.5 Hz pacing, which increased in magnitude with pacing

Taken together, these results illustrate that increasing cell density within meso-ECTs compromises overall contractile stress generation, as measured through uniaxial tensile testing. Further, histological results corroborate that sarcomeres are underdeveloped with increasing hiPSC-CM density. In assessing meso-ECT function, decreasing cell density results in enhanced contractility both at the tissue and hiPSC-CM level with enhanced sarcomere organization. This is likely caused by the increased compaction and developed prestrain experienced by the hiPSC-CMs embedded within the low-density meso-ECTs.

Increasing the Surface Area of ECTs Reveals Scale Impacts Formation and Cell Density Impacts Excitability without Inducing Arrhythmias. The meso-ECTs utilized provide great insight into tissue formation and function at a small scale, which is useful for high-throughput in vitro assessment. However, in scaling up ECTs for preclinical and clinical applicability, the surface area is an important consideration to ensure coverage of the entire injured region of the myocardium. We scale up our meso-ECTs (3×9 mm) into macro-ECTs (8×12 mm), which feature a larger surface area and additional PDMS posts to influence the final shape during compaction. For these experiments, the scope is limited to a lower cell density condition of 15 M/mL, which is chosen as it has been a historical standard, as well as the high-density condition of 50 M/mL. Although the same collagen concentration of 1 mg/mL is initially used in macro-ECT fabrication, due to the high prevalence of macro-ECT breakage in both density conditions, the final collagen concentration is increased to 3.5 mg/mL for all experiments. This collagen concentration also balances structural survival and elastic modulus with force generation when re-tested in the meso-ECTs.

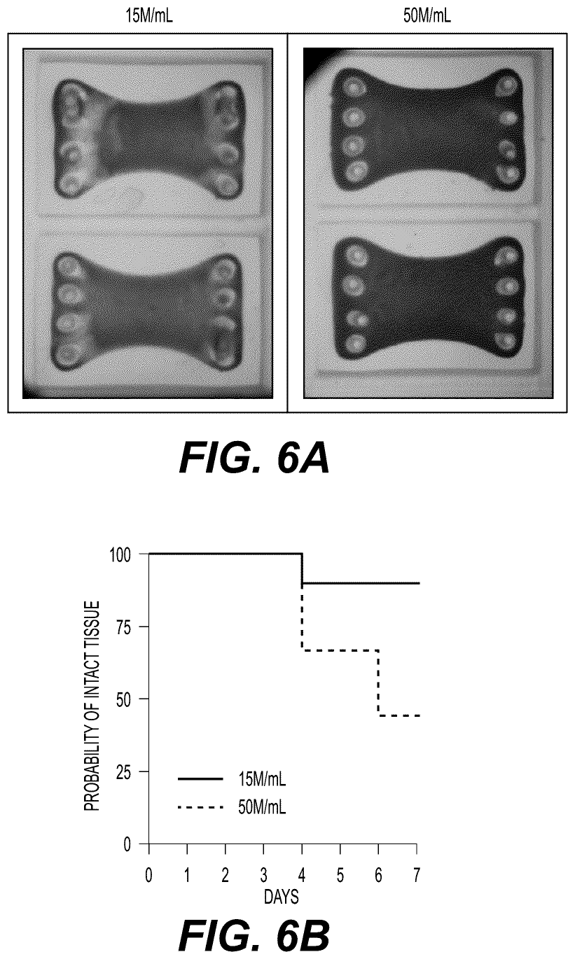

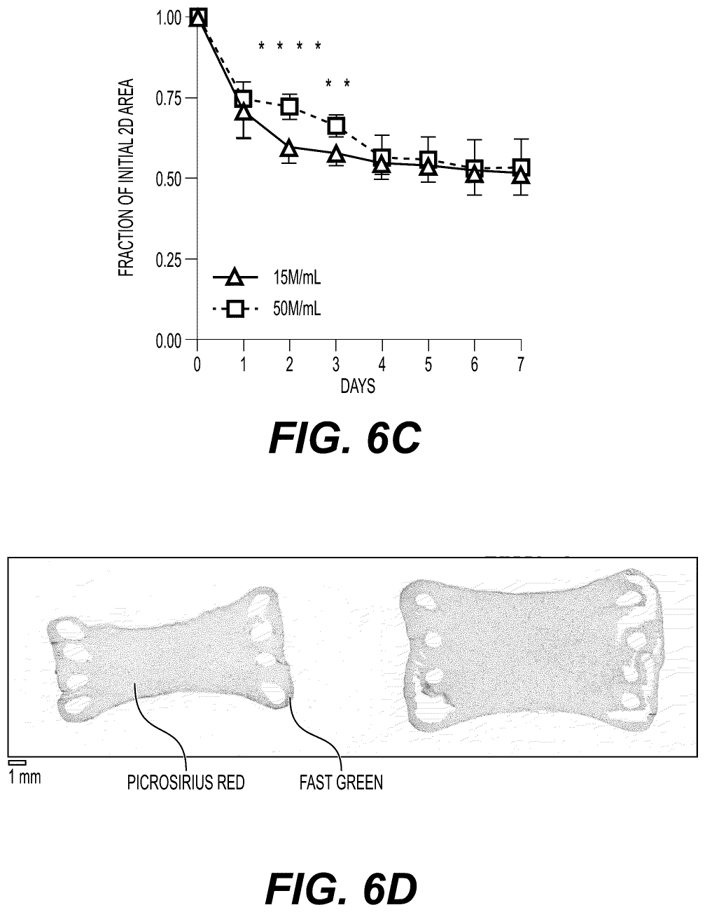

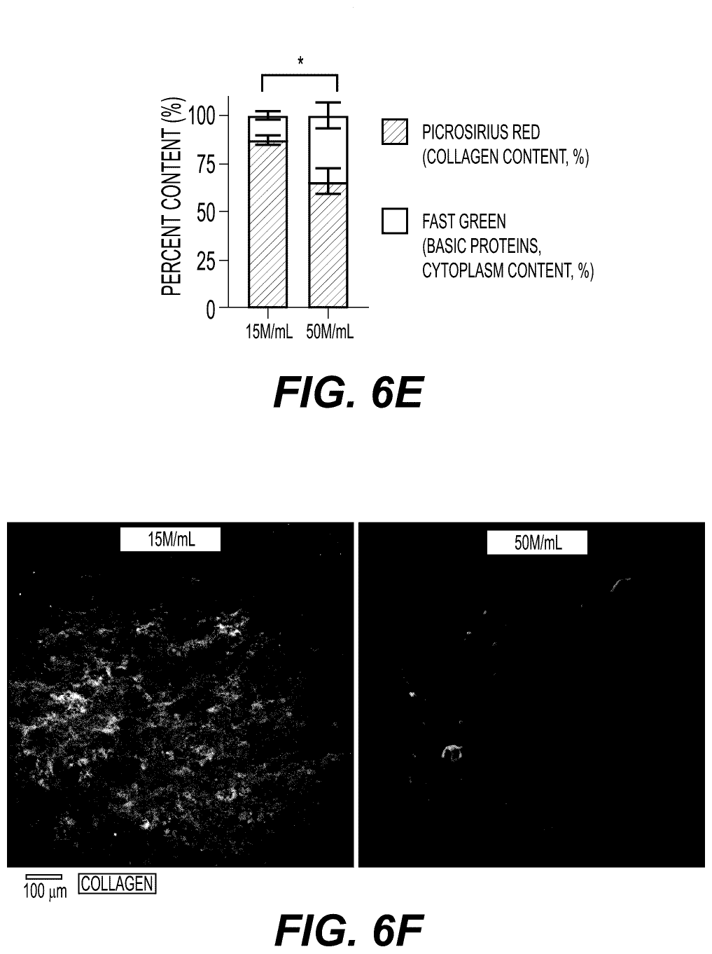

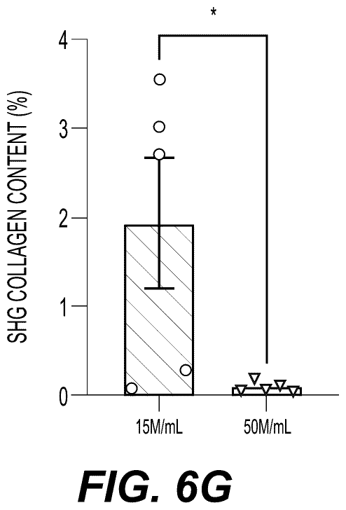

Over 1 week of culture, macro-ECTs with densities of 15 M/mL and 50 M/mL form and compact (FIG. 6A). Patterns of tissue structural survival in culture are comparable to meso-ECTs, with the 15 M/mL group exhibiting higher survival than the 50 M/mL group (FIG. 6B). Macro-ECT structural survival is maintained in both groups until day 4 (15 M/mL: 90.00%; 50 M/mL: 66.67%), with continued fracture seen in the 50 M/mL group down to 44.44% at day 7. Unlike the meso-ECT system, high-density macro-ECT breakage occurs predominately at the posts, suggesting maintained structural internal integrity of the macro-ECT, which has not been seen previously, which may be due to the increased collagen hydrogel concentration utilized. Compaction of macro-ECTs stabilizes by day 4 (15 M/mL: 0.55±0.01; 50 M/mL: 0.57±0.03, fraction of initial area), despite greater compaction at days 2-3 in the 15 M/mL group (FIG. 6C).

ECM deposition and remodeling are evaluated using PRFG (FIGS. 6D-6E) and SHG (FIGS. 6F-6G). Similar to the meso-ECTs, increased cell density in the macro-ECTs correlates with decreased picrosirius red (collagen content) as measured by PRFG (15 M/mL: 77.14±0.99%; 50 M/mL: 58.43±6.57%, % area analyzed) as well as organized collagen measured by SHG imaging (15 M/mL: 1.93+0.72%; 50 M/mL: 0.072+0.02%, % area analyzed). Additionally, the 15 M/mL group exhibits a higher ratio of organized to unorganized collagen content (15 M/mL: 2.21; 50 M/mL: 0.11, measured as SHG signal normalized to PRFG collagen content). Normalization of collagen content to cardiac differentiation batch purity is performed by multiplying the differentiation purity (cTnT+ as assessed by flow cytometry), and quantification is performed to confirm its minimal effect. Compared to the meso-ECT system, collagen content in the macro-ECTs of 15 M/mL and 50 M/mL densities assessed by PRFG increases by 52.0% and 45.2%, respectively, likely due to the 3.5-fold increased collagen hydrogel concentration used in tissue formation. However, organized collagen quantified through SHG imaging decreases in both conditions by 52.58% and 87.88% for the 15 M/mL and 50 M/mL conditions, respectively. The lack of structured collagen within the macro-ECTs may be due to the impact of the altered geometry on the internal tension generated within the tissue. These results suggest increased collagen concentration aids in stabilizing the structural integrity of macro-ECTs during the initial days of tissue formation, but not in prolonged culture. Furthermore, across the meso- and macro-ECT scales, differences in collagen remodeling are observed, likely due to geometric influences.

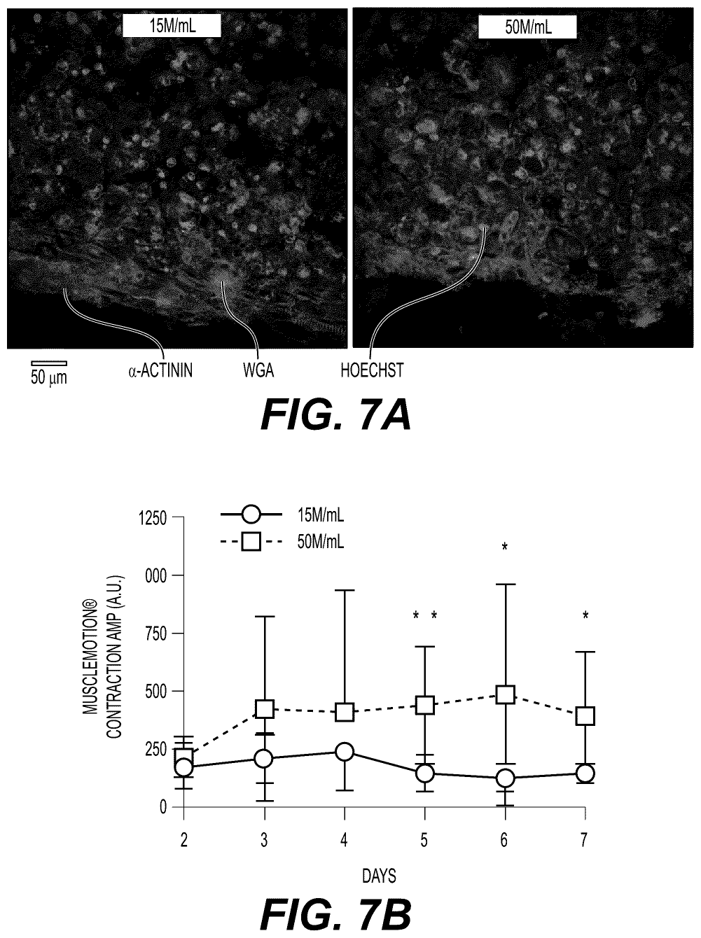

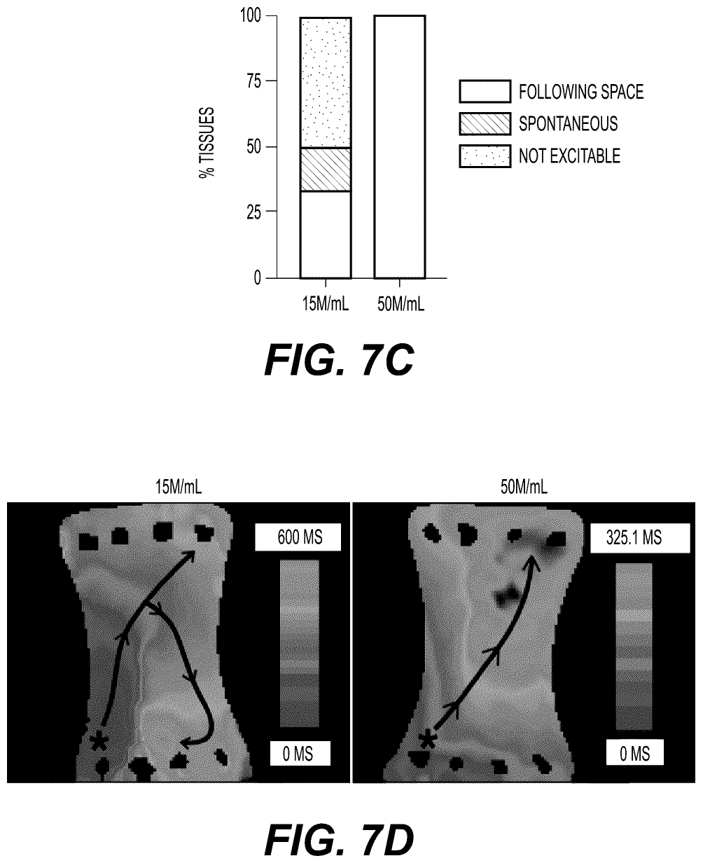

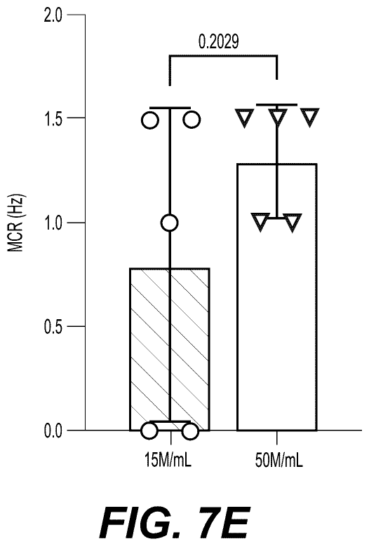

Macro-ECTs are next analyzed to determine the extent of structural development and function. Histological staining illustrated reduced sarcomere organization in the 50 M/mL condition as compared to the 15 M/mL condition (FIG. 7A). However, in both conditions, aligned sarcomeres are limited to the tissue edge, with no significant differences found in sarcomere length (15 M/mL: 1.44±0.04 μm; 50 M/mL: 1.33±0.10 μm). Nuclei size as well as WGA and α-actinin content normalized by the number of nuclei is also calculated, but no significant differences are found between conditions. Similar to the meso-ECTs, nuclear fragmentation, which is a hallmark of cells undergoing apoptosis and necrosis, is not found through the thickness of the macro-ECT, suggesting that cellular viability is maintained. Contractility as assessed by MUSCLEMOTION® shows that the 50 M/mL macro-ECTs has significantly increased force generation compared to the 15 M/mL tissues at day 7, as measured by contraction amplitude (15 M/mL: 150.5+14.60 a.u.; 50 M/mL: 394.5+137.1 a.u., p=0.019) (FIG. 7B).

A critically important aspect of ECT function is its electrical coupling to the host myocardium, which allows uniform propagation of action potentials to assist mechanical function by triggering synchronous contraction across the entire ECT in sync with the host. Arrhythmia generation from implanted hiPSC-CMs is a primary concern in recent literature, particularly with islands of injected (uncoupled) CMs, motivating the assessment of the electrophysiology in large surface area ECTs where reentrant arrhythmias may develop spatially. Macro-ECTs of 15 M/mL and 50 M/mL densities are optically mapped to assess calcium and voltage propagation, during which a stimulation electrode is placed on the corner of the ECTs to trigger a propagating excitation wave (point stimulation). One major difference between the density conditions is the ability of the macro-ECT to follow pace. All tested 50 M/mL macro-ECTs follow the 0.5 Hz point stimulation given. On the other hand, several 15 M/mL macro-ECTs are not excitable by pacing (0.3 mA, 2 ms duration) (50%) or exhibit spontaneous beating (16.66%) that is faster than the set pacing cycle length (FIG. 7C). The activation maps of GCaMP calcium transients in the 15 M/mL condition display nonuniform propagation with conduction block and rotation, forming reentry. In the high-density 50 M/mL macro-ECTs, however, a smooth wavefront without rotation is present (FIG. 7D). Voltage mapping is also performed, with local heterogeneities in the voltage mapping appearing smoothed in the calcium mapping, as expected from EC coupling kinetics. Conduction velocities in the area without conduction block are not significantly different in both GCaMP calcium transients (15 M/mL: 16.70±13.74 x-direction, 24.42±0.003 y-direction, 21.01±4.75 xy-direction; 50 M/mL: 25.55±9.55 x-direction, 26.73±7.11 y-direction, 17.52±3.72 xy-direction) and action potentials (15 M/mL: 8.39±4.64 x-direction, 15.62±2.1 y-direction, 11.74±1.56 xy-direction; 50 M/mL: 5.83±1.68 x-direction, 18.86±4.17 y-direction, 9.892+1.30 xy-direction). In measuring propagation velocity, point stimulation is used to mimic the excitatory signal that the macro-ECT would likely receive from neighboring host cells if it is engrafted and coupled into a host heart. However, field stimulation is also performed to determine the maximum capture rate that can be achieved. In this case, the high-density 50 M/mL macro-ECTs trend toward higher stimulation rates as compared to the 15 M/mL macro-ECTs (15 M/mL: 0.80±0.34 Hz; 50 M/mL: 1.30±0.12 Hz, p=0.209).

Together, the foregoing results show that cell-dense macro-ECTs have a greater ability to follow electrical pacing with little to no arrhythmia generation in vitro. In scaling up the surface area of ECTs, spatial consideration of both contractility and electrical propagation shows encouraging functional results in the highest 50 M/mL macro-ECTs.

Clinical Scale Mega-ECT Enables Delivery of 1 Billion hiPSC-CMs to the Chronically Ischemic Swine Heart. A primary goal of this study is to assess clinical translation of ECTs. Therefore, the fabrication and workflow are scaled up to accommodate an ECT of clinically relevant cell dose and size. Up to this point, smaller-scale ECTs have elucidated how cell density impacts tissue formation and function, reporting some compromises (i.e., decreased compaction and sarcomere organization) and benefits (i.e., reduced arrhythmia) that exist when fabricating cell-dense planar ECTs. Because of this delicate balance between tissue function and density, it becomes necessary to prioritize which properties are essential for therapeutic application, which is why cell dose is chosen to be prioritized. In considering ECTs for regenerative applications, it is hypothesized that compromises in tissue function in vitro immediately after formation can be overcome through in vivo maturation of the ECT after implantation. Thus, a goal is to embed 1 billion hiPSC-CMs within the mega-ECT using a density of 50 M/mL.

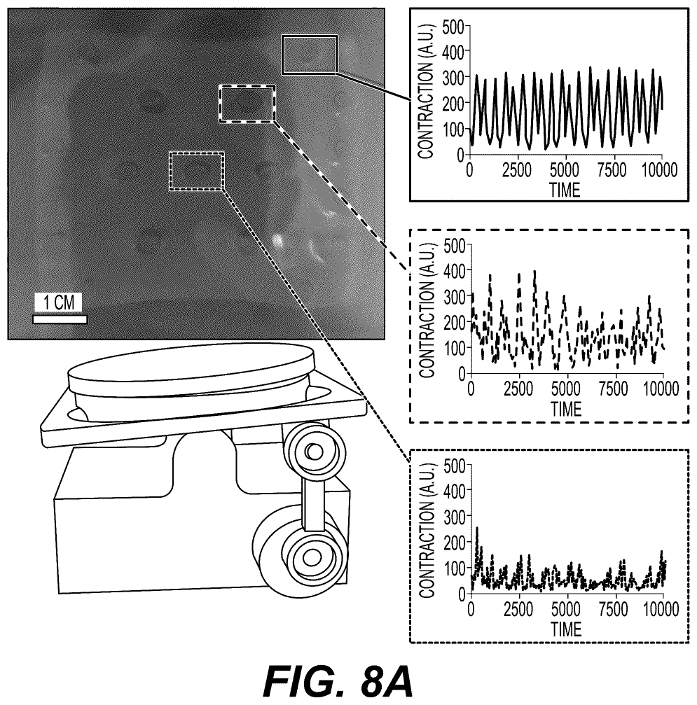

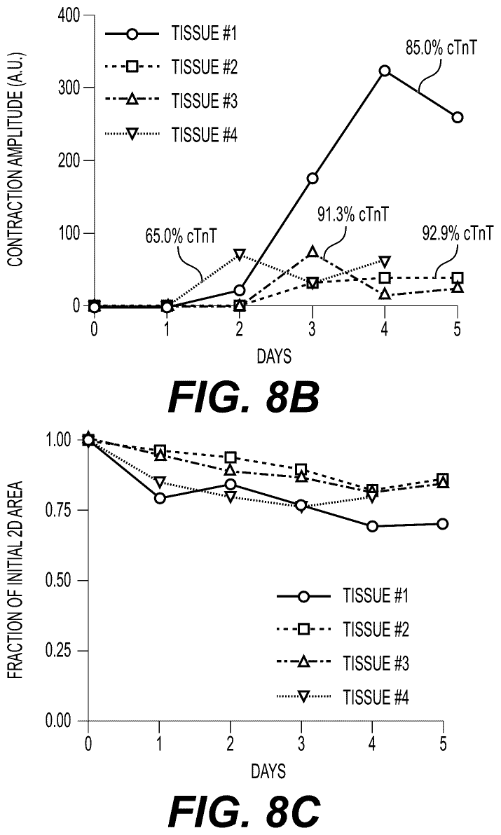

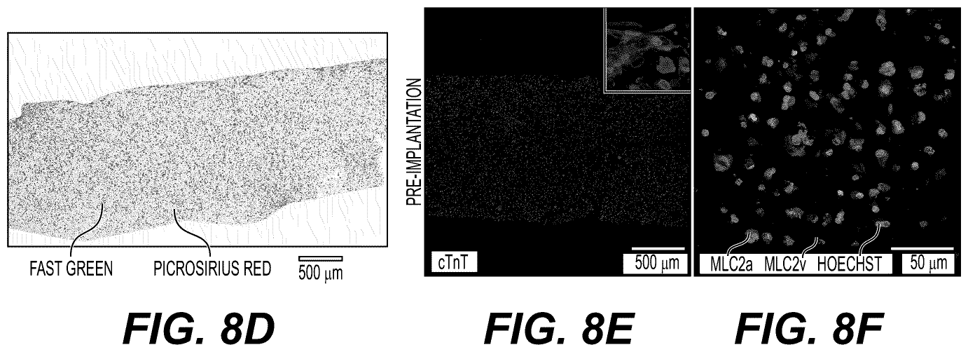

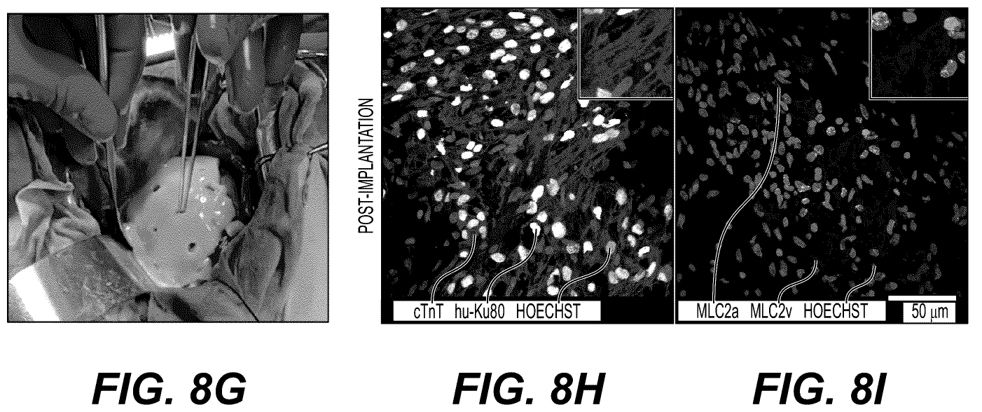

Because of this massive cell quantity, an important aspect to consider during the manufacturing of the mega-ECT is the clinical environment in which this therapy would be employed. The left ventricle (LV) lumen of the human heart at its midpoint is roughly 40 mm in diameter and 90 mm long during diastole, as measured by contrast-enhanced computed tomography. Estimating the LV as a semi-ellipsoid, the maximum surface area of the LV can be calculated to be ˜10,287 mm². Assuming the surgical field of view cuts the LV in half, there is a viable surface area for implantation of less than ˜5144 mm². It was sought to engineer the mega-ECT to maximize this implantable surface area, in order to: (1) cover the infarct as well as part of the healthy myocardium; (2) minimize the necessary cell density; and (3) minimize tissue thickness (measuring ˜1-1.5 mm after compaction). To achieve this, a PDMS mold of dimensions 75 x 65 mm, and therefore a working surface area of 4875 mm², is generated and used for casting the ECT. Utilizing a cell density of 50 M/mL in 20 mL with 3.5 mg/mL collagen hydrogel, mega-ECTs containing 1 billion hiPSC-CMs (FIG. 8A) are fabricated.

To promote nutrient diffusion within the tissue, dynamic culture is employed using a custom rocking plate adapted from NIH 3D, which has been reported in the literature to support the culture of thick (˜1.25 mm) ECTs (FIG. 8A). After 2-3 days of dynamic culture, beating throughout the tissue as analyzed by MUSCLEMOTION® (FIGS. 8A-8B) is observed. Tissue compaction at day 4 is calculated to be 0.78±0.03 (fraction of initial area), which is decreased compared to the 50 M/mL group of the macro-ECTs (0.57±0.03) and meso-ECTs (0.40±0.02) (FIG. 8C). In scaling up, the location of PDMS posts is updated to include internal posts, which stabilize the tissue during culture and further promote nutrient diffusion. Post spacing is informed by earlier work in order to minimally disrupt electrical propagation. For the evaluation of the mega-ECTs during culture, these internal posts are found to be essential, as tissue compaction away from the posts allows visualization contractility throughout the tissue. Immunohistochemical analysis of the mega-ECT at day 5 reveals cells throughout the tissue thickness with no evidence of necrotic patches (see e.g., FIGS. 8D-8E). It is hypothesized that the decreased cell density of the mega-ECT as compared to native myocardium (estimated as ˜108-109 cell/cm²), as well as the dynamic culture conditions implemented, allow for oxygen and nutrient diffusion throughout the thickness of the tissue. PRFG staining is performed with an average collagen content measured to be 84.67±0.69% as well as further quantification from histological staining. Unlike the meso- and macro-ECTs, sarcomere elongation and organization are not observed. It is hypothesized that this lack of organization, as well as the different contractility amplitudes observed throughout the tissue, may be a consequence of the decreased compaction and strain felt by the embedded hiPSC-CMs in this larger format.

As a next step in translation, the mega-ECTs are assessed in a swine model of chronic myocardial ischemia. In this surgical model, chronic ischemia is induced by placing an ameroid constrictor around the left circumflex artery (LCX, FIG. 2B). The slow swelling of the constrictor reduces blood flow and gradually induces ischemia, as opposed to rapid occlusion (often used experimentally), to reflect clinically relevant cases in patients with coronary artery disease. After 4 weeks of ischemia, the mega-ECTs are implanted on the epicardial surface of the swine heart (n=2) to assess technical feasibility. An aggressive immunosuppression regimen is administered daily starting one week prior to ECT implantation to minimize xenograft rejection. The mega-ECTs are structurally robust to allow for successful transportation, surgical handling, and implantation onto the epicardial surface (FIG. 8G) via sutures. It is noted, however, that the mega-ECTs can benefit from a further increase in mechanical integrity to withstand the strong contractile forces of the beating swine heart. Unfortunately, one animal experienced surgical complications (presumed blood clot), resulting in premature death.

Histological analysis to determine hiPSC-CM engraftment is performed 4 weeks after implantation in the surviving swine, revealing persistent human grafts, assessed with the human nuclear marker Ku80 (FIG. 8H, located in the anterior-lateral basal region of the heart). Surviving human grafts feature cardiomyocytes with highly organized sarcomere structures as well as high levels of the mature myosin light chain, MLC2v, compared to the more immature isoform, MLC2a. Comparing this endpoint histology (FIG. 8I, located in the anterior-lateral basal region of the heart) to that of the pre-implant control (FIG. 8F) suggests that significant in vivo maturation of the hiPSC-CMs occurs. In this chronic ischemia model, the scar morphology of the swine successfully implanted with the mega-ECT shows interstitial, diffusive fibrosis through immunohistochemical staining of the LV. Heart rate is monitored daily throughout the study, confirming no occurrence of tachyarrythmias. Together, the foregoing results show successful delivery and engraftment of hiPSC-CM patches in a swine model of chronic myocardial ischemia with no arrhythmia generation. This is believed to be the first implantation of 1 billion hiPSC-CMs within a single ECT in a swine model of MI injury and the first to utilize the chronic ischemia model with PSC-CM delivery.

Despite the importance of scaling up ECTs in terms of cell dose to maximize therapeutic impact as well as size for clinical relevance, the design parameters have not been adequately defined for manufacturing ECTs that can accommodate up to 1 billion CMs for preclinical testing (Table 3) or clinical trials (Table 4). As shown above, scaling up of ECTs needs defining of the design space for engineering implantable ECTs with varying hiPSC-CM density, in part by identifying the criteria, constraints, and challenges of this system. By fabricating ECTs across three length scales, different key aspects of ECT formation and function are studied, demonstrating that scaling up ECTs is a nontrivial and nonlinear challenge. Beginning with meso-ECTs, aspects of formation and function in a higher throughput manner are investigated. The wide range of cell densities (5 M/mL, 15 M/mL, 30 M/mL, 50 M/mL, and 75 M/mL) chosen allows for the comparison of the results to current literature (˜5-15 M/mL; Table 3), while also defining the design limits of the system. High densities are essential to test as they allow the scale-up process to reach 1 billion hiPSC-CMs within a single ECT of reasonable size for implantation on the adult heart. For example, the lowest density condition of 5 M/mL would require a volume of 200 mL to contain 1 billion hiPSC-CMs, an order of magnitude higher than the volume used in our mega-ECT, which is unrealistic to maintain an appropriate tissue surface area and thickness. Therefore, hiPSC-CM densities up to 75 M/mL are evaluated and it is determined that 50 M/mL is a workable solution for creating human-sized ECTs. Through this upscaling effort, 1 billion hiPSC-CMs are embedded within a clinically sized ECT (6.5×7.5 cm). There are few studies investigating scaling ECT to clinically relevant size (>3×3 cm), and these are often limited to low PSC-CM doses (e.g., 4 M and 10 M). In the latter case, for example, ECTs have previously been upscaled from a surface area of 7×7 mm/0.5×106 hiPSC-CMs to 15×15 mm/2 M hiPSC-CMs and further to 3.6×3.6 cm/10 M with an estimated density of ˜0.2-0.3 M hiPSC-CMs/mL, which is more than two orders of magnitude lower than the 50 M hiPSC-CMs/mL utilized in this study. Fabrication of robust, cell-dense ECTs is essential for encapsulation and delivery of therapeutically impactful cell numbers, as well as robust surgical handling and suturing of the tissue during implantation.

TABLE 3
Doses of hiPSC-CMs within ECTs reported in current preclinical
research for cardiac regenerative therapies.

| Research Group | Dose(s)/ECT | Dimensions | | Experiments |
|---|---|---|---|---|
| Eschenhagen | 450M | 50 × 70 | mm | In vivo: healthy swine heart |
| and Weinberger | (hiPSC-CMs) | | | Successful engraftment of hiPSC- |
| | | | | CMs |
| | 4.5M, 8.5M, | 15 × 25 | mm | In vivo: guinea pig cryoinjury model |
| | 12M, 15M | | | Cell dose dependence on |
| | (hiPSC-CMs) | | | remuscularization of scar |
| | | | | Improvement in LV FAS at high |
| | | | | dose (1 2M) |
| | | | | Importance of hiPSC-CM maturity |
| | | | | for engraftment |
| Zhang | 4M/ECT | 40 × 20 | mm | In vivo: swine I/R model of MI |
| | 8M/2-ECTs | | | 2 ECTs implanted over infarct |
| | (hiPSC-CMs) | | | Improved EF compared to sham |
| | | | | and non-cellular implants |
| | | | | Decreased LVEDV and infarct size |
| | | | | compared to sham and non-cellular |
| | | | | implants |
| Bursae | 0.5M/1M | 7 × 7 | mm | In vitro/in vivo: |
| | (hiPSC-CMs) | | | Increased stress generation, force |
| | | | | per CM, velocity of action potential |
| | | | | at lower cell density |
| | | | | No adverse effect on host electrical |
| | | | | function |
| | 2M/10M | 15 × 15 | mm | In vitro: |
| | (hiPSC-CMs) | 36 × 36 | mm | Scale up for proof of concept |
| Coulombe | 7-10M | 18 × 14 | mm | In vivo: rat I/R model of MI |
| | (hiPSC-CMs) | | | Trend illustrating improved viable |
| | | | | engrafted area of ECTs loaded with |
| | | | | proangiogenic factors |
| | | | | Correlated with improved |
| | | | | vascularization of ECT as well as |
| | | | | infarct area with proangiogenic |
| | | | | loading of ECTs |
| | | | | Improved FS of ECTs with |
| | | | | angiogenic factor loading |
| | | | | compared to sham |
| Keller | 2.64-5.28M | 15 × 15 | mm | In vivo: rat model of MI |
| | (hiPSC-CMs) | | | Improved EF, CO, and regional LV |
| | | | | radial and longitudinal strain at 4 |
| | | | | weeks |
| | 10.56M | 30 × 30 | mm | In vitro: |
| | (hiPSC-CMs) | | | Scale up for proof of concept |
| Zimmerman | 2.5M/ECT | ~5 × 10 | mm | In vivo: rat PL model of MI |
| | 12.5M/5-ECT | | | Generating multiloop ECTs by |
| | (Neonatal rat | | | stacking 5 single loop ECTs |
| | CMs) | | | Induced systolic wall thickening |
| | | | | Improved FAS compared to |
| | | | | controls |

MI = myocardial infarction, LV = left ventricle, I/R = ischemia-reperfusion, PL = permanent ligation, FAS = fractional area shortening, FS = fractional shortening, LVEDV = left ventricle end diastolic volume, EF = ejection fraction, CO = cardiac output; M = million.

TABLE 4
Doses of hiPSC reported in current human clinical trials for cardiac regenerative therapies.

| Trial Number | Dose | Disease | Description | Phase | Start/End |
|---|---|---|---|---|---|
| NCT03759405 | Not | CHF | Autologous iPS- | 2-3 | December2022/ |
| | specified | | CMs via vein | | December 2024 |
| | | | transplantation | | |
| NCT03763136 | 200M | CHF | Injection of | 1-2 | October 2021/ |
| | | | allogenic hPSC- | | July 2023 |
| | | | CM during | | |
| | | | coronary artery | | |
| | | | bypass surgery | | |
| NCT04396899 | Not | HFrEF | Engineered heart | 1-2 | February2020/ |
| | specified | (EF <35%) | muscle | | October 2024 |
| NCT05068674 | 10M/150M/ | Chronic | Dose tolerance | 1 | March 2022/ |
| | 300M | ischernic LV | study of hESC- | | October 2025 |
| | | dysfunction | CMs | | |
| NCT04982081 | 100M/400M | Congestive HF, | hiPSC-CMs | 1 | September 2021/ |
| | | CVD, dilated | catheter injection | | July 2023 |
| | | Cardiomyopathy | | | |
| NCT046963 28/ | (3) sheets | Ischernic | Allogenic iPSC- | 1 | December 2019/ |
| jRCT | of 33M/ | cardiomyopathy | CM within a cell | | May 2023 |
| 2053190081 | sheet | | sheet | | |

CHF = chronic heart failure, CVD = cardiovascular disease, HFrEF = heart failure with reduced ejection fraction, MI = myocardial infarction/ischemia, LV = left ventricle, EF = ejection fraction; M = million.

Through a holistic examination of these results, both hiPSC-CM density and scale are identified as important factors that influence ECT formation and function in vitro. Within the small-scale meso-ECTs, density impacted the environment within the tissues, leading to changes in the structural and mechanical development of the tissue, aligning with other reports in the literature. For example, the hiPSC-CM dose is varied from just 0.5 M to 1 M within ECTs, finding decreased stress generation (21.0%) as well as force per CM (43.7%) and decreased conduction velocity (212.6%). Biomechanical cues such as passive stretching and loading provide improved ECT structural organization, contractility, and matrix deposition. In upscaling to the planar macro-ECT, mechanical cues shift compaction dynamics as maintaining a maximal surface area of the tissue becomes more important. Unlike the meso-ECTs, both compaction and mechanical function of the macro-ECTs in vitro converges regardless of density, suggesting the significant influence of tissue scale on tissue formation and function. Further functional benefits are also noted in the cell-dense macro-ECT condition. For example, of significant clinical importance and translation is the ability to study how the action potentials and calcium transient signals propagate throughout the larger surface area of upscaled ECTs. Arrhythmia generation from PSC-CMs is of great clinical relevance, as recent studies have shown that injecting PSC-CMs has resulted in life-threatening arrhythmia generation in preclinical models, hypothesized to be due to the spontaneous excitation of the implanted CMs. At the macro-ECT level, however, increased hiPSC-CM density enables better pacing control as well as uniform wavefront propagation, suggesting a reduced risk of arrhythmia. These findings allow fabrication of an even larger mega-ECT composed of 1 billion hiPSC-CMs for implantation in a large animal model of chronic myocardial ischemia. Upon implantation of the mega-ECT in vivo on the epicardial surface of the swine heart, significant maturation of hiPSC-CMs occurs over the next 4 weeks. This data supports the notion that ECTs for applications as regenerative therapies may need to prioritize cell quantity and structural integrity of the tissue while allowing functional maturation to occur after implantation.

In this work, scale-specific aspects of ECT formation and function are leveraged to define biomanufacturing parameters for creating high-density ECTs. With high-throughput small-scale ECTs (meso-ECTs, 3×9 mm), the significant impact of hiPSC-CM density on tissue formation and function over 7 days of culture in vitro is reported, allowing the narrowing of the range of tested cell densities and hydrogel composition in order to support ECT formation. Scaling up to a larger, planar surface area (macro-ECTs, 8×12 mm), the impact of hiPSC-CM density on electrical signal propagation and tissue arrhythmogenesis after 7 days of in vitro culture is additionally evaluated. Cell-dense macro-ECTs are able to follow point stimulation pacing without arrhythmogenesis better than the low-density condition. Finally, a single mega-ECT (65×75 mm) composed of 1 billion hiPSC-CMs is fabricated and implanted into a swine model of chronic myocardial ischemia. The technical surgical feasibility of this therapeutic approach is successfully demonstrated. Engraftment and maturation of iPSC-CMs without incidence of arrhythmia over 4 weeks in vivo is demonstrated. Through this work, hiPSC-CM density as well as ECT size and shape have been identified as considerations in scaling up to clinically relevant cell doses. Taken together, this study refines the many variables involved in ECT biomanufacturing at a clinically relevant scale that influence ECT formation and function, considers what may be important for in vivo vs. in vitro endpoints, and identifies the ways to leverage these variables to create ECT therapies with high translation potential for heart regeneration.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An engineered cardiac tissue (ECT) composition comprising cardiomyocytes (CMs) and having a CM density of about 5 million CMs/mL to about 75 million CMs/mL.

2. The composition of claim 1, having a CM density of about 15 million CMs/mL to about 75 million CMs/mL.

3. The composition of claim 1, having a CM density of about 50 million CMs/mL.

4. The composition of claim 1, comprising up to about 1 billion CMs.

5. The composition of claim 1, wherein the cardiomyocytes are human induced pluripotent stem-cell derived cardiomyocytes (hiPSC-CMs) or human embryonic stem-cell derived cardiomyocytes (hESC-CMs).

6. The composition of claim 1, further comprising a hydrogel substantially comprised of collagen.

7. The composition of claim 1, further comprising a hydrogel substantially comprised of collagen hydrogel in a density of about 1 mg/mL to about 8mg/mL.

8. The composition of claim 1, further comprising a hydrogel substantially comprised of collagen in a density of about 3.5 mg/mL.

9. The composition of claim 1, further comprising cardiac fibroblasts (CFs).

10. The composition of claim 8, wherein the number of CFs is about 5% to about 50% of the number of CMs.

11. The composition of claim 8, wherein the number of CFs is about 5% of the number of CMs.

12. The composition of claim 1, wherein the ECT composition has a volume that is surgically implantable in a living animal.

13. The composition of claim 12, wherein the ECT composition has a volume ranging from about 25 microliter (μL) to about 25 milliliter (mL).

14. A mold comprising posts, and further comprising the ECT composition of claim 1.

15. A method for generating heart tissue in a subject, the method comprising implanting the ECT composition of claim 1 in the subject in need thereof.

16. The method of claim 11, wherein the subject suffers from cardiovascular disease (CVD).

17. The method of claim 11, where in the subject has had at least one episode of myocardial infarction (MI).

18. A method of forming an engineered cardiac tissue (ECT) construct suitable for covering at least part of the heart of a subject, the method comprising the steps of:
(a) providing a cell solution comprising human induced stem cell differentiated cardiomyocytes (hiPSC-CMs) and human fibroblasts wherein the number of human fibroblasts is about 5% to about 25% of the number of hiPSC-CMs;
(b) providing a hydrogel substantially comprised of collagen;
(c) preparing a casting mix by combining the cell solution of step (a) with the hydrogel of step (b) such that the hiPSC-CMs are dispersed throughout and suspended within the hydrogel;
(d) incubating the mixture of step (c) in a mold system having a defined shape to form an engineered tissue having a shape defined by the surface of the mold;
(e) culturing the engineered tissue up to about 7 days; and
(f) separating the tissue construct from the molding system to obtain the ECT construct suitable for covering at least part of the heart of a subject.

19. A method of forming an engineered cardiac tissue (ECT) construct suitable for covering at least part of the heart of a subject, the method comprising the steps of:
(a) providing a cell solution comprising human induced stem cell differentiated cardiomyocytes (hiPSC-CMs) and human fibroblasts;
(b) providing a hydrogel substantially comprised of collagen wherein the hydrogel has collagen in a density of about 3.5 mg/ml;
(c) preparing a casting mix by combining the cell solution of step (a) with the hydrogel of step (b) such that the hiPSC-CMs are dispersed throughout and suspended within the hydrogel;
(d) incubating the mixture of step (c) in a mold system having a defined shape to form an engineered tissue having a shape defined by the surface of the mold;
(e) culturing the engineered tissue up to about 7 days; and
(f) separating the tissue construct from the molding system to obtain the ECT construct suitable for covering at least part of the heart of a subject.

20. A method of forming an engineered cardiac tissue (ECT) construct suitable for covering at least part of the heart of a subject, the method comprising the steps of:
(a) providing a cell solution comprising human induced stem cell differentiated cardiomyocytes (hiPSC-CMs) and human fibroblasts;
(b) providing a hydrogel substantially comprised of collagen;
(c) preparing a casting mix by combining the cell solution of step (a) with a hydrogel of step (b) in a ratio ranging from about 1:1 to about 2:1 such that the hiPSC-CMs are dispersed throughout and suspended within the hydrogel;
(d) incubating the mixture of step (c) in a mold system having a defined shape to form an engineered tissue having a shape defined by the surface of the mold;
(e) culturing the engineered tissue up to about 7 days; and
(f) separating the tissue construct from the molding system to obtain the ECT construct suitable for covering at least part of the heart of a subject.
